SEQUENCE LISTING (2 sequences)

SEQ ID NO: 1 - DNA, 29 bp, Artificial Sequence
  Sry3
atggagggccatgtcaagcgccccatgaa

SEQ ID NO: 2 - DNA, 30 bp, Artificial Sequence
  Sry5
ttgctggtttttggagtacaggtgtgcagc

(19) United States
(12) Patent Application Publication
(10) Pub. No.: US 2014/0331339 A1
(43) Pub. Date: Nov. 6, 2014

(54) Transgenic Non-Human Assay Vertebrates, Assays and Kits

(71) Applicant: Kymab Limited, Cambridge (GB)

(72) Inventors: Allan Bradley, Cambridge (GB); E-Chiang Lee, Cambridge (GB); Qi Liang, Cambridge (GB)

(73) Assignee: Kymab Limited, Cambridge (GB)

(21) Appl. No.: 14/263,176

(22) Filed: Apr. 28, 2014

Related U.S. Application Data
(60) Provisional application No. 61/819,057, filed on May 3, 2013.

(51) Int. Cl. A01K 67/027; C12N 15/85; A61K 49/00

(52) U.S. Cl. CPC A01K 67/0275; A61K 49/0004; C12N 15/8509; C12N 2015/8518; A01K 2227/105; A01K 2267/03
USPC 800/3; 800/14; 800/18

(57) ABSTRACT

The invention provides Assay Vertebrates comprising a human antigen or epitope knock-in for testing antibodies comprising human variable regions and generated in a related Antibody-Generating Vertebrate. The invention also provides kits and methods involving these vertebrates and antibodies. The invention provides for superior assay models and assay methods of chimaeric and other test antibodies comprising human variable regions.

This application claims the benefit of provisional application U.S. 61/819,057, filed May 3, 2013, the entirety of which is hereby incorporated by reference.

The present invention relates inter alia to non-human vertebrates useful as assay models, as well as kits and methods of making and using such vertebrates for testing chimaeric antibodies.

### BACKGROUND

Animal models are widely used in research, giving many advantages such as providing for versatile experimentation in a way that is accessible to the research community and in a way that is more ethically acceptable than research on humans. Such models are routinely used to assess the toxicology, pharmacokinetics, efficacy and other characteristics of drugs in vivo prior to administration to humans in clinical trials.

Knock-in and knock-out animal models have been produced in which the effect of removing or adding a single gene can be assessed in vivo.

Additionally, using embryonic stem cell (ES cell) technology, the art has provided non-human vertebrates, such as mice, bearing transgenic chimaeric antibody loci from which human or chimaeric antibodies can be generated in vivo following challenge with human antigen. Such antibodies usefully bear human variable regions.

It is desirable to provide improved non-human vertebrates as models for assaying such human and chimaeric antibodies in vivo in the presence of the human antigen.

### SUMMARY OF THE INVENTION

To this end, the present invention provides: —

A method of assaying a test antibody comprising human variable regions that bind to a human epitope, wherein the antibody is isolated from a first transgenic non-human vertebrate (eg, a mouse or rat) (Antibody-Generating Vertebrate) following immunisation with an antigen bearing said human epitope (with optional subsequent derivatisation or maturation of said antibody), the vertebrate comprising one or more transgenic antibody loci encoding said variable regions, and the transgenic vertebrate having an immune system comprising proteins encoded by an immune gene repertoire, said immune gene repertoire comprising said transgenic antibody loci, the method comprising

(a) Providing a second transgenic non-human vertebrate (eg, mouse or rat) (Assay Vertebrate) that is a modified version of said first transgenic non-human vertebrate, wherein the Assay Vertebrate comprises

(i) An immune system comprising proteins encoded by substantially the same immune gene repertoire as the Antibody-Generating Vertebrate;

(ii) A genome comprising a knock-in of said human epitope, so that the Assay Vertebrate is capable of expressing an antigen bearing said human epitope; and

(iii) Optionally wherein said genome has a knock-out of an endogenous non-human vertebrate epitope that is an orthologue or homologue of said human epitope, wherein said Assay Vertebrate cannot express an antigen bearing said endogenous epitope;

(b) Introducing said antibody into the Assay Vertebrate; and

(c) Assaying the effect or behaviour of said antibody in said Assay Vertebrate.

The invention also provides: —

A non-human (eg, mouse or rat) Assay Vertebrate comprising

(i) One or more transgenic antibody loci encoding human variable regions;

(ii) An immune system comprising proteins encoded by an immune gene repertoire, said immune gene repertoire comprising said transgenic antibody loci;

(iii) A genome comprising a knock-in of a human epitope, so that the Assay Vertebrate is capable of expressing an antigen bearing said human epitope; and

(iv) A genome knock-out of the endogenous non-human vertebrate epitope that is an orthologue or homologue of said human epitope, wherein said Assay Vertebrate cannot express an antigen bearing said endogenous epitope; and

(v) Optionally a test antibody inside said Assay Vertebrate, wherein the antibody comprises human variable regions that can bind said human epitope, said antibody having been generated in an Antibody-Generating Vertebrate as defined above (with optional subsequent derivatisation or maturation to produce said antibody).

The invention also provides: —

A method of assaying a test antibody comprising human variable regions that bind to a human epitope, wherein the antibody is isolated from a first transgenic non-human vertebrate (eg, a mouse or rat) (Antibody-Generating Vertebrate) following immunisation with an antigen bearing said human epitope (with optional subsequent derivatisation or maturation of said antibody), the vertebrate comprising one or more transgenic antibody loci encoding said variable regions, the method comprising

(a) Providing a second transgenic non-human vertebrate (eg, mouse or rat) (Assay Vertebrate) that is a modified version of said first transgenic non-human vertebrate, wherein the Assay Vertebrate has substantially the same genome as the Antibody-Generating Vertebrate, with the exception that
(i) the Assay Vertebrate genome comprises a knock-in of said human epitope, so that the Assay Vertebrate is capable of expressing an antigen bearing said human epitope; and
(ii) Optionally wherein said genome has a knock-out of an endogenous non-human vertebrate epitope that is an orthologue or homologue of said human epitope, wherein said Assay Vertebrate cannot express an antigen bearing said endogenous epitope;

(b) Introducing said antibody into the Assay Vertebrate; and

(c) Assaying the effect or behaviour of said antibody in said Assay Vertebrate.

The invention also provides: —

A non-human (eg, mouse or rat) Assay Vertebrate comprising

(i) One or more transgenic antibody loci encoding human variable regions;

(iii) A genome comprising a knock-in of a human epitope, so that the Assay Vertebrate is capable of expressing an antigen bearing said human epitope; and

(iv) A genome knock-out of the endogenous non-human vertebrate epitope that is an orthologue or homologue of said human epitope, wherein said Assay Vertebrate cannot express an antigen bearing said endogenous epitope; and

(v) Optionally a test antibody inside said Assay Vertebrate, wherein the antibody comprises human variable regions that can bind said human epitope, said antibody having been generated in an Antibody-Generating Vertebrate as defined in any one of claims 1 to 6, 15 and 16 (with optional subsequent derivatisation or maturation to produce said antibody).

The invention also provides methods of making non-human Assay Vertebrates.

The non-human vertebrates and methods of the invention enable the generation and testing of human antibody variable regions against human epitopes in a way that eliminates or minimises background variability between the antibody and the immune setting of the system used to test the antibody. By generating and testing antibodies in substantially the same immune background, complicating issues of immune reaction against the test antibody are eliminated or minimised in the assay vertebrate. Furthermore, testing and antibody generation can be matched for human epitopes of interest and improved assays can be performed by harnessing Assay Vertebrate in vivo tolerisation against human antigen of interest.

### BRIEF DESCRIPTION OF THE FIGURES

FIG. 1: The progressive changes in morphology of cultured blastocysts (taken from “Manipulating the Mouse Embryo”, 3^{rd} Edition, A Nagy et al, Cold Spring Harbor Laboratory Press, 2003; FIG. 8.2 of that text).

FIG. 2A: Characteristic and illustrative ES cell morphology (taken from “Manipulating the Mouse Embryo”, 3^{rd} Edition, A Nagy et al, Cold Spring Harbor Laboratory Press, 2003; FIG. 8.4 of that text).

FIG. 2B: Photograph showing ES cells generated according to the example below (KX01.3 cells shown).

FIG. 3: A schematic representation of a precise gene knock-out method.

FIGS. 4A and 4B: Schematic representations of a precise gene knock-out method.

### DETAILED DESCRIPTION OF THE INVENTION

The invention provides an assay vertebrate and a method of assaying a test antibody comprising human variable regions that specifically bind to a human epitope, which in one embodiment is an epitope on a human target. For example, an entire human target is used or alternatively a portion of such a human target is used optionally fused to a heterologous protein moiety, wherein said portion comprises the human epitope of interest. In one example, the heterologous protein moiety is a transmembrane domain optionally with an associated intracellular domain. The heterologous protein moiety can be a mouse protein moiety (eg, a mouse protein domain, eg, a mouse transmembrane domain optionally with an associated mouse intracellular domain) or an antibody Fc region (eg, a mouse or human Fc). In one embodiment, the human epitope is provided on an extracellular domain of a human target. The human target is, for example, selected from the group consisting of growth factors, cytokines, cytokine receptors, enzymes, co-factors for enzymes and DNA binding proteins. In one example, the human epitope is provided on a multi-subunit protein (eg, an oligomer) such as a receptor (eg, a dimeric or trimeric receptor) or multimeric ligand. The multisubunit protein can, for example, comprise a first subunit bearing the human epitope and one or more second subunits (eg, mouse or human subunits). It is advantageous to harbour the human epitope in the context of protein domains of the non-human vertebrate species (eg, mouse or rat), such as a non-human vertebrate (eg, mouse or rat) transmembrane domain and/or intracellular signalling domain of a protein target in which the human epitope is present on an extracellular domain of the protein target. This allows for proper anchoring and signalling in the non-human vertebrate (Assay Vertebrate) when the human epitope is bound by the test antigen. Thus, this embodiment accommodates the knocked-in human epitope in a context useful for efficient and representative assaying within the Assay Vertebrate.

The antibodies described herein can be of any format provided that they comprise human variable regions. For example, the present invention is applicable to of 4-chain antibodies, where the antibodies each contain 2 heavy chains and 2 light chains. Alternatively, the invention can be applied to H2 antibodies (heavy chain antibodies) bearing human V regions and which are devoid of CH1 and light chains (equivalent in respects to Camelid H2 antibodies: see, eg, Nature. 1993 Jun. 3; 363(6428):446-8; Naturally occurring antibodies devoid of light chains; Hamers-Casterman C, Atarhouch T, Muyldermans S, Robinson G, Hamers C, Songa E B, Bendahman N, Hamers R). These antibodies function to specifically bind antigen, such antibodies being akin to those found in the blood of Camelidae (eg, llamas, camels, alpacas). Such antibodies with human VH pairs can be synthetically produced to provide therapeutic and prophylactic medicaments (eg, see WO1994004678, WO2004041862, WO2004041863). Transgenic mice also can produce such heavy chain antibodies and the in vivo production of the antibodies allows the mouse's immune system to select for human VH-VH pairings, sometimes selecting for such pairings in which mutations have been introduced in vivo by the mouse to accommodate the pairing (W02010109165A2). Thus, in an embodiment of the present invention, the heavy chain transgene is devoid of a CH1 gene segment and the genome comprises no functional antibody light chain locus. Alternatively, the test antibody is an antibody fragment, eg, Fab or Fab₂, which comprises a constant region and human variable regions.

Throughout this text, and with application to any configuration, aspect, embodiment or example of the invention, the term “endogenous” (eg, endogenous constant region) in relation to a non-human vertebrate or cell indicates that the constant region a type of constant region that is normally found in the vertebrate or cell (as opposed to an exogenous constant region whose sequence is not normally found in such a vertebrate or cell).

The test antibody is isolated from a first transgenic non-human vertebrate (eg, a mouse or rat) (Antibody-Generating Vertebrate) following immunisation with an antigen bearing said human epitope. The skilled person will be familiar with routine methods and protocols for immunising with antigen, eg, using prime and boost immunisation protocols. A suitable protocol is RIMMS (see Hybridoma 1997 August; 16(4):381-9; “Rapid development of affinity matured monoclonal antibodies using RIMMS”; Kilpatrick et al). The Antibody-Generating Vertebrate comprises one or more transgenic antibody loci encoding said variable regions. Suitable non-human vertebrates (eg, mice or rats) are known in the art, and by way of example reference is made to WO2011004192, U.S. Pat. No. 7,501,552, U.S. Pat. No. 6,673,986, U.S. Pat. No. 6,130,364, WO2009/076464 and U.S. Pat. No. 6,586,251, the disclosures of which are incorporated herein by reference in their entirety. In one example, the Antibody-Generating Vertebrate is a mouse having a 129 mouse genetic background. In one example, the Assay Vertebrate is a mouse having a 129 mouse genetic background, for example the same genetic background as the Antibody-Generating Vertebrate but with the human target knock-in. In one example, the Antibody-Generating Vertebrate is a mouse having an AB2.1 mouse genetic background. In another example, the Antibody-Generating Vertebrate is a mouse having a genetic background of a mouse strain selected from a 129 strain, C57BL/6N, C57BL/6J, 129S5, 129S7 or 129Sv or the genetic background of a cell selected from a JM8, AB2.1 or AB2.2 cell. In one example, the background is a mouse 129 strain×C57BL/6 strain cross, eg, 129S7×C57BL/6 or 129S5×C57BL/6. In an example, the background is a mouse B6 background or a B6-derived background.

Examples of suitable 129 strains are as follows (see also http://www.informatics.jax.org/mgihome/nomen/strain_{—}129.shtml)

### 129 Strain Designation

129P1

129P2

129P3

129X1

129S1

129S1

129S2

129S4

129S5

129S6

129S7

129S8

129T1

129T2

129T2

The transgenic vertebrate has an immune system comprising proteins encoded by an immune gene repertoire (eg, an endogenous immune gene repertoire), said immune gene repertoire comprising said transgenic antibody loci and genes for immune system function (eg, providing an immune response to immunisation of the Antibody-Generating Vertebrate to the human target epitope). In one embodiment, the immune gene repertoire is an endogenous immune gene repertoire (ie, endogenous to the strain of non-human vertebrate used). For example, when the Antibody-Generating Vertebrate is a mouse having a genetic background of a mouse strain or cell selected from 129, C57BL/6N, C57BL/6J, JM8, AB2.1, AB2.2, 12955, 12957 or 129Sv, the mouse has an immune gene repertoire provided by said genetic background and said transgenic antibody loci. Thus, the skilled person can choose the appropriate starting strain, cell or species (eg, the same cell line or cells separated by no more than 5, 4, 3, 2 or 1 generation) for generating both the Antibody-Generating Vertebrate and Assay Vertebrate, and in doing so the desired immune gene repertoire is provided for both Vertebrates. In one embodiment, the immune gene repertoire is that of a wild-type 129, C57BL/6, B6 or other mouse strain or mouse cell disclosed herein, with the exception that the mouse genome comprises a transgenic IgH locus (optionally in homozygous state) comprising a human variable region (with human VH, D and JH gene segments) operatively connected upstream of (5′ of) a mouse constant region and optionally endogenous mouse heavy chain expression is inactive. In an example, the genome also comprises a transgenic Igκ locus (optionally in homozygous state) comprising a human variable region (with human Vκ and Jκ gene segments) operatively connected upstream of (5′ of) a mouse constant region and optionally endogenous mouse kappa chain expression is inactive. In an example, the genome also comprises a transgenic Igλ locus (optionally in homozygous state) comprising a human variable region (with human Vλ and Jλ gene segments) operatively connected upstream of (5′ of) a mouse constant region and optionally endogenous mouse lambda chain expression is inactive. Thus, in one embodiment, the vertebrate of the invention comprises a wild-type 129, C57BL, B6 or other mouse strain genome with the exception that mouse heavy chain (and kappa and/or lambda chain) expression has been inactivated, the genome comprises said transgenic Ig loci and an endogenous target knock-out (and optionally also a human target knock-in) as per the invention. Thus, endogenous regulatory and control mechanisms and proteins functional to produce and regulate immune responses in the vertebrate are retained for production of chimaeric antibody chains having human variable regions in response to immunisation.

In one embodiment the Antibody-Generating Vertebrate is a genetic parent or grandparent of the Assay Vertebrate. In one embodiment, the Vertebrates are related as (a) siblings, (b) parent and child, (c) parent and grandchild, (d) cousins or (e) uncle/aunt and nephew/niece. This is achieved by breeding (crossing) vertebrates in a method using the genome of the Antibody-Generating Vertebrate. To this end, the invention also provides methods for making the Assay Vertebrate, and this is explained in further detail below.

In one embodiment, the Assay Vertebrate is derived from a somatic cell of said Antibody-Generating Vertebrate; optionally wherein the Assay Vertebrate is derived from an IPS cell (induced pluripotent stem cell) that is derived from said Antibody-Generating Vertebrate. Reference is made to WO2007069666, WO2008118820, WO2008124133, WO2008151058, WO2009006997 and WO2011027180, which provide guidance on IPS technology and suitable methods, the disclosures of which are incorporated herein in their entirety. The IPS cells can also be directly generated (ie, without need for breeding) from other somatic cells from non-human vertebrates (eg, mice) carrying the antibody transgenes using standard methods. A worked example of ES cell derivation is provided in the Examples below.

The method of the invention comprises the step of providing a second transgenic non-human vertebrate (eg, mouse or rat) (Assay Vertebrate) that is a modified version of said first transgenic non-human vertebrate (ie, Antibody-Generating Vertebrate), wherein the Assay Vertebrate comprises
(i) An immune system comprising substantially the same (or the same) immune gene repertoire as the Antibody-Generating Vertebrate;
(ii) A genome comprising a knock-in of said human epitope, so that the Assay Vertebrate is capable of expressing an antigen bearing said human epitope; and
(iii) Optionally wherein said genome has a knock-out of an endogenous non-human vertebrate epitope that is an orthologue or homologue of said human epitope, wherein said Assay Vertebrate cannot express an antigen bearing said endogenous epitope.

In one aspect, the Antibody-Generating Vertebrate and Assay Vertebrate have identical or substantially identical genomes with the exception that the Assay Vertebrate genome comprises said knock-in.

Thus, when the Antibody-Generating Vertebrate is a mouse, the Assay Vertebrate is also a mouse, eg, a mouse of the same genetic background as the Antibody-Generating Vertebrate (except of the knock-in and optional knock-out). Thus, in one embodiment, the Antibody-Generating Vertebrate and Assay Vertebrates have a 129 genetic background. In another embodiment, the Vertebrates have an AB2.1 genetic background. In yet another embodiment, the Vertebrates have a C57BL background. In a further embodiment, the Vertebrates have a JM8 background.

Thus, when the Antibody-Generating Vertebrate is a rat, the Assay Vertebrate is also a rat, eg, a rat of the same genetic background as the Antibody-Generating Vertebrate (except of the knock-in and optional knock-out).

In one aspect, the Antibody-Generating Vertebrate and Assay Vertebrate genomes comprise said knock-out. This is useful, for example, when the endogenous orthologue/homologue epitope or target protein is structurally or epitopically similar to the human target or epitope. By knocking-out the orthologue/homologue expression, test antibodies of interest are generated only to the human epitope/target that is injected into the Antibody-Generating Vertebrate, and isolation of antibodies that are raised against the orthologue/homologue (ie, wrong target) is avoided. Advantageously, this target expression profile is reproduced in the Assay Vertebrate when the orthologue/homologue is knocked-out in that model too.

Thus, in an embodiment, the Antibody-Generating Vertebrate has a knock-out of the epitope that is an orthologue or homologue of said human epitope. Additionally or alternatively, in an embodiment, the Assay Vertebrate has a knock-out of the epitope that is an orthologue or homologue of said human epitope.

Additionally, in the present invention, both the Antibody-Generating and Assay Vertebrates produce antibodies with human variable regions and constant regions of the same type (eg, both Vertebrates are mice and the transgenic loci encode chimaeric antibodies having human variable regions and mouse constant regions, eg, constant regions endogenous to the strain of mouse used to generate the Vertebrates). Thus, the test antibody that is injected into the Assay Vertebrate is not seen as foreign to that Vertebrate and is not substantially immunologically rejected or attacked by the Assay Vertebrate's immune system. Moreover, the Assay Vertebrate expresses the human epitope or target as a “self” antigen, and thus the mouse's immune system has been tolerised to this antigen during development of the Assay Mouse immune system. This minimises interference in vivo of the assay by any anti-human epitope/target antibodies produced by the Assay Vertebrate itself, thereby enabling more effective and accurate assessment of the effect and/or behaviour of the test antibody following introduction into the Assay Vertebrate. Thus, the invention matches the immune characteristics of the Vertebrates and test antibodies and harnesses the Assay Vertebrate's ability to tolerise in the presence of the knocked-in human epitope or target antigen of interest. This provides for superior in vivo pre-clinical and clinical assessment of test antibodies than has been possible previously.

In one embodiment, the transgenic antibody loci of the Assay Vertebrate are human antibody loci comprising human variable and constant region gene segments. Optionally, the test antibody is a human antibody comprising human variable and constant regions and this is administered to the Assay Vertebrate of this embodiment. Optionally, the test antibody is generated in an Antibody-Generating Vertebrate in which the transgenic antibody loci are human antibody loci comprising human variable and constant region gene segments. Thus, the model animals and test antibodies are matched, as per the present invention.

The skilled person will be familiar with conventional techniques for manipulating non-human vertebrate (eg, mouse or rat) genomes in embryonic stem cells (ES cells), as well as application to knock-in genes (ie, insert a desired gene into the genome of the ES cell) and knock-out genes (ie, delete a gene from the genome of an ES cell). Tools such as site-specific recombination (eg, using Cre/Lox, Frt/Flp, Dre/Rox and others) and homolgous recombination are standard. By way of example and background, reference is made to New England Journal of Medicine 2007 Dec. 13; 357(24):2426-9; “Knock out, knock in, knock down—genetically manipulated mice and the Nobel Prize”; Manis J P, which explains that: for the construction of knock-out mice, a gene-targeting vector can be constructed to delete a specific exon of a gene in embryonic stem cells. Several kilobases of DNA on either side of the target gene are cloned around a drug-selection marker. After the cloned DNA (targeting vector) is introduced into the stem cells, positive and negative drug selection occurs in culture. For example, a targeting vector is constructed with loxP sequences flanking the positive drug-selection gene. Cre recombinase can delete the DNA sequence between the loxP sites, thereby deleting a specific gene in the embryonic stem cells. Knock-in mice can be generated insertion of DNA (eg, human target DNA) of interest with or without concomitant deletion of an endogenous DNA (eg, the endogenous target orthologue/homologue). For the latter, the gene-targeting strategy is similar to that used for knock-out mice, except that a replacement DNA is exchanged with the endogenous DNA. Cre-loxP strategies can delete most traces of the targeting vector. Once the desired stem-cell clone is selected, it is injected into a blastocyst (eg, of a mouse C57BL, JM8 or 129 strain), which is implanted into the uterus of a foster mother (eg, a mouse mother). If the gene-targeted stem cells contribute to germ cells in the chimaeric mice, subsequent offspring will harbour the gene-targeted mutation (germ-line transmission has been achieved). Optional subsequent breeding can be carried out between the offspring to breed the knock-in and knock-out to homozygosity, as is standard.

In an Assay Vertebrate of the invention according to any aspect, the Vertebrate comprises a second human knock-in nucleotide sequence, the second sequence encoding a second human protein. Optionally, the second human protein is part of a cascade comprising the first human epitope/target. Optionally the first and second human epitope/targets are related as human ligand and receptor, eg, human CD40 ligand and human CD40. Immunisation of the Assay-Generating Vertebrate can be with human CD40 ligand or CD40 and a resultant isolated antibody (or derivative thereof) can be tested in the Assay Vertebrate bearing a knock in of both human CD40 ligand and human CD40. Thus, in one embodiment of the Assay Vertebrate, the first epitope is human CD40 ligand or human CD40.

The test antibody isolated from the Antibody-Generating Vertebrate can be introduced (eg, injected) into the Assay Vertebrate in unmodified form. Alternatively, the test antibody can be derivatised, eg, by the addition (such as by chemical conjugation) of a label or toxin, PEG or other moiety, prior to introduction into the Assay Vertebrate. Derivatisation is useful, for example, when it is desirable to add an additional functionality to the drug to be developed from the test antibody. For example, for cancer indications it may be desirable to add additional moieties that assist in cell-killing. In another embodiment, the variable regions of the antibody isolated from the Antibody-Generating Vertebrate are affinity matured in vivo or in vitro (eg, by phage display, ribosome display, yeast display, etc) and a matured test antibody is introduced into the Assay Vertebrate. In another embodiment, the constant regions of the antibody isolated from the Antibody-Generating Vertebrate are mutated in vivo or in vitro (eg, by random or directed, specific mutation and optional selection by phage display, ribosome display, yeast display, etc) and a matured test antibody is introduced into the Assay Vertebrate. The constant region may be mutated to ablate or enhance Fc function (eg, ADCC). Thus, derivatised and/or mutated antibodies are considered “test antibodies” in this context. The constant region may be humanised (ie, where a chimaeric antibody is isolated from the Assay-Generating Vertebrate having human variable regions and non-human constant regions, the latter may be exchanged for human constant regions and the resultant human antibody introduced into the Assay Vertebrate).

The method of the invention entails assaying the effect or behaviour of said antibody in said Assay Vertebrate. For example, said assaying is assay of one or more selected from the group consisting of: pharmacodynamics of said antibody (or a metabolite or derivative thereof produced by the Assay Vertebrate), pharmacokinetics of said antibody (or a metabolite or derivative thereof produced by the Assay Vertebrate), activity of said antibody (or a metabolite or derivative thereof produced by the Assay Vertebrate), clearance of said antibody (or a metabolite or derivative thereof produced by the Assay Vertebrate), distribution of said antibody (or a metabolite or derivative thereof produced by the Assay Vertebrate), toxicology of said antibody (or a metabolite or derivative thereof produced by the Assay Vertebrate), a physico-chemical characteristic or effect of said antibody (or a metabolite or derivative thereof produced by the Assay Vertebrate), a binding characteristic of said antibody (or a metabolite or derivative thereof produced by the Assay Vertebrate), a biological characteristic or effect of said antibody (or a metabolite or derivative thereof produced by the Assay Vertebrate), a physiological characteristic or effect of said antibody (or a metabolite or derivative thereof produced by the Assay Vertebrate), a pharmaceutical characteristic or effect of said antibody (or a metabolite or derivative thereof produced by the Assay Vertebrate), and interaction of said antibody (or a metabolite or derivative thereof produced by the Assay Vertebrate) with another protein or substance inside the Assay Vertebrate. Such assays are well known to the skilled person. For example, said assaying is assay of immunogenicity of the test antibody.

The invention provides a non-human (eg, mouse or rat) Assay Vertebrate comprising

(i) One or more transgenic antibody loci encoding human variable regions;

(ii) An immune system comprising proteins encoded by an immune gene repertoire, said immune gene repertoire comprising said transgenic antibody loci;

(iii) A genome comprising a knock-in of a human epitope, so that the Assay Vertebrate is capable of expressing an antigen bearing said human epitope; and

(iv) A genome knock-out of the endogenous non-human vertebrate epitope that is an orthologue or homologue of said human epitope, wherein said Assay Vertebrate cannot express an antigen bearing said endogenous epitope; and

(v) Optionally a test antibody inside said Assay Vertebrate, wherein the antibody comprises human variable regions that can bind said human epitope, said antibody having been generated in an Antibody-Generating Vertebrate as defined above (with optional subsequent derivatisation or maturation to produce said antibody).

The various aspects of the Vertebrates, epitopes, targets, knock-in, knock-out, test antibodies, immune gene repertoire and all other aspects described herein apply to this configuration of the invention that provides the Assay Vertebrate per se.

In one embodiment, the transgenic antibody loci of any aspect of the invention are according to the loci described in any of WO2011004192, U.S. Pat. No. 7,501,552, U.S. Pat. No. 6,673,986, U.S. Pat. No. 6,130,364, WO2009076464 and U.S. Pat. No. 6,586,251, the disclosures of which are incorporated herein by reference in their entirety. In one example, the Antibody-Generating Vertebrate comprises
(a) A heavy chain locus comprising one or more human heavy chain V gene segments, one or more human heavy chain D gene segments and one or more human heavy chain JH gene segments upstream of an endogenous non-human vertebrate (eg, endogenous mouse or rat) constant region (eg, Cmu and/or Cgamma);
(b) A kappa light chain locus comprising one or more human kappa chain V gene segments, and one or more human kappa chain Jk gene segments upstream of an endogenous non-human vertebrate (eg, endogenous mouse or rat) kappa constant region; and optionally
(c) A lambda light chain locus comprising one or more human lambda chain V gene segments, and one or more human lambda chain Jλ gene segments upstream of a lambda constant region; and
(d) Wherein the Vertebrate is capable of producing chimaeric test antibodies following rearrangement of said loci and immunisation with the human epitope or target.

Optionally endogenous heavy and kappa chain expression is inactive. In an embodiment, endogenous lambda chain expression is also inactive.

Alternatively or additionally, the Assay Vertebrate comprises
(a) A heavy chain locus comprising one or more human heavy chain V gene segments, one or more human heavy chain D gene segments and one or more human heavy chain JH gene segments upstream of an endogenous non-human vertebrate (eg, endogenous mouse or rat) constant region (eg, Cmu and/or Cgamma);
(b) A kappa light chain locus comprising one or more human kappa chain V gene segments, and one or more human kappa chain Jk gene segments upstream of an endogenous non-human vertebrate (eg, endogenous mouse or rat) kappa constant region; and optionally
(c) A lambda light chain locus comprising one or more human lambda chain V gene segments, and one or more human lambda chain Jλ gene segments upstream of a lambda constant region; and
(d) Wherein the Assay Vertebrate is capable of producing chimaeric antibodies;
(e) Optionally wherein said Assay Vertebrate loci comprise substantially the same repertoire of human antibody gene segments as said Antibody-Generating Vertebrate loci (optionally with rearrangement of the loci in one or both of said Vertebrates).
(f) Optionally endogenous heavy and kappa chain expression is inactive. In an embodiment, endogenous lambda chain expression is also inactive.

The invention provides an Antibody-Generating Vertebrate as herein described, optionally as part of a kit also comprising a test antibody as herein described.

The invention provides an assay kit comprising an Antibody-Generating Vertebrate and Assay Vertebrate as defined herein and optionally a test antibody (or derivative thereof). In one example, the test antibody has been isolated from said Antibody-Generating Vertebrate or a relative thereof that is no more than 5, 4, 3, 2 or 1 generations way from the Assay Vertebrate and comprises substantially the same immune gene repertoire.

A kit of the invention, in an aspect, comprises instructions instructing administration of a test antibody with an Assay Vertebrate as herein described.

The invention provides a method of generating a non-human Assay Vertebrate (eg, a mouse or rat) for assaying the effect or behaviour of a test antibody comprising human variable regions and which binds a human epitope, the method comprising
(a) Providing an ES cell derived from an Antibody-Generating Vertebrate whose genome encodes said test antibody, the Antibody-Generating Vertebrate comprising one or more transgenic antibody loci encoding antibodies comprising human variable regions, and the Antibody-Generating Vertebrate having an immune system comprising proteins encoded by an immune gene repertoire, said immune gene repertoire comprising said transgenic antibody loci;
(b) Introducing into the genome of the ES cell a nucleotide sequence encoding said human epitope and optionally knocking-out of the genome an endogenous non-human vertebrate epitope that is an orthologue or homologue of said human epitope; and
(c) Developing a non-human child vertebrate from said modified ES cell, wherein an Assay Vertebrate is obtained that expresses said human epitope; and
(d) Optionally producing a progeny of said Assay Vertebrate, wherein said progeny comprises substantially the same immune gene repertoire as said Assay Vertebrate in addition to the human epitope knock-in (and optional knock-out) and optionally the progeny is homozygous for said nucleotide sequence encoding the knocked-in human epitope (and optionally also homozygous for the knocked-out orthologous or homologous epitope).

The Examples illustrate a method of obtaining an ES cell derived from an Antibody-Generating Vertebrate.

The invention provides a method of generating a non-human Assay Vertebrate (eg, a mouse or rat) for assaying the effect or behaviour of a test antibody comprising human variable regions and which binds a human epitope, the method comprising

(a) Obtaining a non-human vertebrate child ES cell whose genome is a genetic cross between (i) the genome of a first genetic parent that is a non-human Antibody-Generating Vertebrate whose genome encodes said test antibody, the Antibody-Generating Vertebrate comprising one or more transgenic antibody loci encoding antibodies comprising human variable regions, and the Antibody-Generating Vertebrate having an immune system comprising proteins encoded by an immune gene repertoire, said immune gene repertoire comprising said transgenic antibody loci and (ii) the genome of a second genetic parent that is a non-human vertebrate of the same species (optionally the same strain or cell background, eg, of mouse strain 129, C57BL/6N, C57BL/6J, JM8, AB2.1, AB2.2, 129S5, 129S7 or 129Sv) as said Antibody-Generating Vertebrate, the second parent having an immune system encoded by substantially the same immune gene repertoire as the first parent;

(b) Producing in vitro a modified child ES cell with a knock-in of the human epitope by introducing into the genome of the child ES cell a nucleotide sequence encoding said human epitope and optionally knocking-out of the genome an endogenous non-human vertebrate epitope that is an orthologue or homologue of said human epitope; and

(c) Developing a non-human child vertebrate from said modified child ES cell, wherein an Assay Vertebrate is obtained that expresses said human epitope; and

(d) Optionally producing a progeny of said Assay Vertebrate by genetic crossing, wherein said progeny comprises substantially the same immune gene repertoire as said Assay Vertebrate in addition to the human epitope knock-in (and optional knock-out).

The first (and optionally also the second) parent is, in one aspect, any Antibody-Generating Vertebrate described herein. Step (a) can be performed, for example, using breeding between parents to achieve the genetic cross in a resulting embryo. The non-human child ES cell can be generated from the embryo (eg, blastocyst stage) using any standard technique for ES cell generation. For example, reference is made to Proc Natl Acad Sci 1997 May 27; 94(11):5709-12; “The origin and efficient derivation of embryonic stem cells in the mouse”; Brook F A & Gardner R L, the disclosure of which is incorporated herein by reference. Other standard ES cell-generating techniques can be used. See also the illustrative, non-limiting Example below.

Knock-in and knock-out technology has been discussed above, and any of these methods can be used to effect step (b).

The skilled person conversant with ES cell technology will readily know how to develop a child from a transgenic ES cell that has been manipulated in vitro. For example, a non-human ES cell obtained in step (b) is implanted into a donor blastocyst (eg, a blastocyst of the same strain of vertebrate as the ES cell). The blastocyst is then implanted into a foster mother where it develops into a child (an Assay Vertebrate). In this way, a plurality of children can be developed, each from a respective modified child ES cell. Siblings can be bred together to achieve crosses providing one or more resultant Assay Vertebrates that are homozygous for the human knock-in (and optional knock-out). Alternatively, an Assay Vertebrate that is heterozygous for the knock-in (and also for the optional knock-out) can be provided. Homozygous or heterozygous Assay Vertebrates can be used to assay a test antibody in the method of the invention.

In one example, the first and second genetic parents (a) (i) and (ii) are of the same non-human vertebrate (eg, mouse or rat) strain or cell background.

In one example, the first and second genetic parents are related as (a) siblings, (b) parent and child, (c) parent and grandchild, (d) cousins or (e) uncle/aunt and nephew/niece.

The invention also provides a method of generating a non-human Assay Vertebrate (eg, a mouse or rat) for assaying the effect or behaviour of a test antibody comprising human variable regions and which binds a human epitope, the method comprising

(a) Obtaining a non-human vertebrate child ES cell from a somatic cell (optionally wherein the ES cell is an IPS cell) of a non-human Antibody-Generating Vertebrate whose genome encodes said test antibody, the Antibody-Generating Vertebrate comprising one or more transgenic antibody loci encoding antibodies comprising human variable regions, and the Antibody-Generating Vertebrate having an immune system comprising proteins encoded by an immune gene repertoire (eg, an endogenous immune gene repertoire), said immune gene repertoire comprising said transgenic antibody loci;

(b) Producing a modified child ES cell with a knock-in of the human epitope by introducing into the genome of the child ES cell a nucleotide sequence encoding said human epitope and optionally knocking-out of the genome an endogenous non-human vertebrate epitope that is an orthologue or homologue of said human epitope; and

(c) Developing a non-human child vertebrate from said modified child ES cell, wherein an Assay Vertebrate is obtained that expresses said human epitope; and

(d) Optionally producing a progeny of said Assay Vertebrate by genetic crossing, wherein said progeny comprises substantially the same immune gene repertoire as said Assay Vertebrate in addition to the human epitope knock-in (and optional knock-out).

The Antibody-Generating Vertebrate used in this method is, in one embodiment, any Antibody-Generating Vertebrate described herein. Step (a) can be performed, for example, using breeding between an Antibody-Generating Vertebrate and a second vertebrate of the same species (eg, another Antibody-Generating Vertebrate with substantially the same immune repertoire and/or substantially the same genetic background) to produce a resulting embryo. Mouse embryo fibroblasts can be generated from the embryo and then IPS cells generated using any standard technique. For example, reference is made to Proc Natl Acad Sci; 2011 Oct. 11; “Rapid and efficient reprogramming of somatic cells to induced pluripotent stem cells by retinoic acid receptor gamma and liver receptor homolog 1”; Wang et al, the disclosure of which is incorporated herein by reference. Other standard IPS-generating techniques can be used.

Knock-in and knock-out technology has been discussed above, and any of these methods can be used to effect step (b).

In one embodiment, the IPS cell is a mouse embryonic fibroblast cell.

### Human Target & Epitope DNA

The DNA encoding the human target or epitope can be from any suitable source, eg, obtained by cloning the DNA from a blood or tissue sample of a human donor. In one embodiment, a cDNA is used that is encodes the human epitope or target. In another embodiment, genomic DNA is used, eg, the gene for the human target. In one example, the coding sequence for the human target is used, together with the endogenous human signal sequence (if present) and promoter (and optionally any enhancer of the gene).

Human DNA is readily obtainable from commercial and academic libraries, eg, Bacterial Artificial Chromosome (BAC) libraries containing human DNA. Examples are the Human RPCI-11 and -13 libraries (Osoegawa et al, 2001—see below; http://bacpac.med.buffalo.edu/11framehmale.htm) and also the “CalTech” Human BAC libraries (CalTech Libraries A, B, C and/or D, http://www.tree.caltech.edu/lib_status.html).

### CalTech Human BAC Library D:

See: http://www.ncbi.nlm.nih.gov/clone/library/genomic/16/

The Hiroaki Shizuya laboratory at the California Institute of Technology has developed three distinct human BAC libraries (obtainable from Open Biosystems). The Cal Tech B (CTB) and Cal Tech C (CTC) libraries together represent a genomic coverage of 15×. The Cal Tech D (CTD) library represents a 17× coverage of the human genome. Whole collections as well as individual clones are available. Detailed information on the construction of the libraries can be found at http://informa.bio.caltech.edu/idx_www_tree.html.

### Library Summary

Library Name: CalTech human BAC library D

Library Abbreviation: CTD

Organism: Homo sapiens

Distributors: Invitrogen, Open Biosystems

Vector type(s): BAC

# clones Clone DB: 226,848

# end sequences Clone DB: 403,688

# insert sequences Clone DB: 3,153

# clones with both ends sequenced: 153,035

### Library Details

| DNA Source: | Sex | Cell type | |
|---|---|---|---|
| | male | Sperm | |
| Library | | | Vector Cloning |
| Construction | Library segment | Vector Name | Site (s) |
| | 1 | pBeloBACII | HindIII |
| DNA Source: | Sex | Cell type | |
| | 2-5 | pBeloBACII | EcoRI |
| Library | | | |
| Statistics | Library segment | Avg Insert (kb) | Plate Range (s) |
| | 1 | 129 | 2001 to 2423 |
| | 2 | 202 | 2501 to 2565 |
| | 3 | 182 | 2566 to 2671 |
| | 4 | 142 | 3000 to 3253 |
| | 5 | 166 | 3254 to 4869 |

### RPCI-11 BACs

### REFERENCES

Osoegawa K, Mammoser A G, Wu C, Frengen E, Zeng C, Catanese J J, de Jong P J; Genome Res. 2001 March; 11(3):483-96; “A bacterial artificial chromosome library for sequencing the complete human genome”;
Osoegawa, K., Woon, P. Y., Zhao, B., Frengen, E., Tateno, M., Catanese, J. J, and de Jong, P. J. (1998); “An Improved Approach for Construction of Bacterial Artificial Chromosome Libraries”; Genomics 52, 1-8;
http://bacpac.chori.org/hmale11.htm, which describes the BACs as follows

### RPCI-11 Human Male BAC Library

The RPCI-11 Human Male BAC Library (Osoegawa et al., 2001) was constructed using improved cloning techniques (Osoegawa et al., 1998) developed by Kazutoyo Osoegawa. The library was generated by Kazutoyo Osoegawa. Construction was funded by a grant from the National Human Genome Research Institute (NHGRI, NIH) (#1R01RG01165-03). This library was generated according to the new NHGRI/DOE “Guidance on Human Subjects in Large-Scale DNA Sequencing”.

Male blood was obtained via a double-blind selection protocol. Male blood DNA was isolated from one randomly chosen donor (out of 10 male donors) and partially digested with a combination of EcoRI and EcoRI Methylase. Size selected DNA was cloned into the pBACe3.6 vector between the EcoRI sites. For Segment 5, the same male donor DNA was partially digested with MboI, size selected, and ligated into the pTARBAC1 cloning vector at the BamHI sites. The ligation products were transformed into DH10B electrocompetent cells (BRL Life Technologies). The library has been arrayed into 384-well microtiter dishes and also gridded onto 22×22 cm nylon high density filters for screening by probe hybridization.

### The RPCI Human Male BAC Library:

| | | | | | | Empty |
|---|---|---|---|---|---|---|
| | Cloning | | Plate | Total | Total | Wells |
| Segment | Vector | DNA | Numbers | Plates | Clones | (Total) |
| 1 | pBACe3.6 ⁽¹⁾ | Male | 1-288 | 288 | 108,499 | 2,093 |
| 2 | pBACe3.6 ⁽¹⁾ | Male | 289-576 | 288 | 109,496 | 1,096 |
| 3 | pBACe3.6 ⁽¹⁾ | Male | 577-864 | 288 | 109,657 | 935 |
| 4 | pBACe3.6 ⁽¹⁾ | Male | 865-1152 | 288 | 109,382 | 1,210 |
| 5 | pTARBAC1 ⁽²⁾ | Male | 1153-1440 | 288 | 106,763 | 3,289 |
| Total Library | | | 1-1440 | 1440 | 543,797 | 9,163 |

⁽¹⁾ donor DNA EcoRI partially digested
⁽²⁾ donor DNA MboI partially digested

| | | Non- | | | |
|---|---|---|---|---|---|
| | | Recom- | Non- | | |
| | | binant | Recom- | | |
| | Empty Wells | Clones | binant | Insert Size | Genomic |
| Segment | (%) | (Total) | Clones (%) | (average) | Coverage |
| 1 | 1.9 | approx. | 1.7 | 164 Kbp | 5.8X |
| | | 1800 | | | |
| 2 | 1.0 | approx. | 0.5 | 168 Kbp | 6.0X |
| | | 550 | | | |
| 3 | 0.8 | approx. | 1.0 | 181 Kbp | 6.7X |
| | | 1100 | | | |
| 4 | 1.1 | approx. | 1.0 | 183 Kbp | 6.8X |
| | | 1100 | | | |
| 5 | 3.5 | approx. | 0.5 | 196 Kbp | 6.9X |
| | | 530 | | | |
| Total | 1.7 | approx. | 0.9 | 178 Kbp | 32.2X |
| Library | | 5080 | | | |

The average insert size has been determined by Pulsed Field Gel Electrophoresis analysis of clones randomly chosen from plates from each segment.

### BAC Availability

The RP11 BACs are available for purchase from Invitrogen (see http://tools.invitrogen.com/content/sfs/manuals/bac_clones_man.pdf).

Vectors, such as BACs or PACs, can be manipulated in vitro by standard Molecular Biology techniques, for example recombineering (see http://www.genebridges.com; EP129142 and EP1204740). For example, recombineering can be used to create vectors in which a nucleotide sequence coding for a human target or epitope of interest is flanked by one or more sequences, such as homology arms or site-specific recombination sites (eg, lox, frt or rox). The homology arms are, in one embodiment, homologous to, or identical to, stretches of DNA from the genome of the non-human vertebrate to be used to generate the Assay Vertebrate. Vectors created in this way are useful for performing homologous recombination (see, eg, U.S. Pat. No. 6,638,768, the disclosure of which is incorporated herein by reference) in a method of precisely inserting the human DNA into the non-human vertebrate genome (eg, to precisely replace the orthologous or homologous DNA in the vertebrate genome).

Other useful DNA- and genome-manipulation techniques are readily available to the skilled person, including technologies described in U.S. Pat. No. 6,461,818 (Baylor College of Medicine), U.S. Pat. No. 6,586,251 (Regeneron) and WO2011044050 (eg, see Examples).

Techniques for constructing non-human vertebrates and vertebrate cells whose genomes comprise a transgene, eg, a transgenic antibody locus containing human V, J and optionally D regions are well known in the art. For example, reference is made to WO2011004192, U.S. Pat. No. 7,501,552, U.S. Pat. No. 6,673,986, U.S. Pat. No. 6,130,364, WO2009/076464 and U.S. Pat. No. 6,586,251, the disclosures of which are incorporated herein by reference in their entirety.

All nucleotide coordinates for the mouse are from NCBI m37, April 2007 ENSEMBL Release 55.37h for the mouse C57BL/6J strain. Human nucleotides are from GRCh37, February 2009 ENSEMBL Release 55.37 and rat from RGSC 3.4 Dec. 2004 ENSEMBL release 55.34w.

In one embodiment in any configuration of the invention, the Antibody-Generating Vertebrate and/or the Assay Vertebrate is a non-human mammal. In one embodiment in any configuration of the invention, the Antibody-Generating Vertebrate and/or the Assay Vertebrate is a mouse, rat, rabbit, Camelid (eg, a llama, alpaca or camel) or shark.

In one aspect the transgenic antibody loci comprise human V, D and/or J coding regions placed under control of the host regulatory sequences or other (non-human, non-host) sequences. In one aspect reference to human V, D and/or J coding regions includes both human introns and exons, or in another aspect simply exons and no introns, which may be in the form of cDNA.

Alternatively it is possible to use recombineering, or other recombinant DNA technologies, to insert a non human-vertebrate (e.g. mouse) promoter or other control region, such as a promoter for a V region, into a BAC containing a human Ig region. The recombineering step then places a portion of human DNA under control of the mouse promoter or other control region.

The invention also relates to a cell line (eg, ES or IPS cell line) which is grown from or otherwise derived from cells or a Vertebrate as described herein, including an immortalised cell line. The cell line may be immortalised by fusion to a tumour cell to provide an antibody producing cell and cell line, or be made by direct cellular immortalisation.

In one aspect the non-human vertebrate of any configuration of the invention is able to generate a diversity of at least 1×10⁶ different functional chimaeric antibody sequence combinations.

Optionally in any configuration of the invention the constant region is endogenous to the Vertebrate and optionally comprises an endogenous switch. In one embodiment, the constant region comprises a Cgamma (Cγ) region and/or a Smu (Sμ) switch. Switch sequences are known in the art, for example, see Nikaido et al, Nature 292: 845-848 (1981) and also WO2011004192, U.S. Pat. No. 7,501,552, U.S. Pat. No. 6,673,986, U.S. Pat. No. 6,130,364, WO2009/076464 and U.S. Pat. No. 6,586,251, eg, SEQ ID NOs: 9-24 disclosed in U.S. Pat. No. 7,501,552. Optionally the constant region comprises an endogenous S gamma switch and/or an endogenous Smu switch.

In one aspect the test antibodies have a part of a non-human vertebrate host constant region sufficient to provide one or more effector functions seen in antibodies occurring naturally in a host vertebrate, for example that they are able interact with Fc receptors, and/or bind to complement.

Reference to a chimaeric antibody or antibody chain having a non-human vertebrate constant region herein therefore is not limited to the complete constant region but also includes chimaeric antibodies or chains which have all of the host constant region, or a part thereof sufficient to provide one or more effector functions. This also applies to non-human Vertebrates and cells and methods of the invention in which human variable region DNA may be inserted into the host genome such that it forms a chimaeric antibody chain with all or part of a host (endogenous) constant region. In one aspect the whole of a host non-human vertebrate constant region is operably linked to human variable region DNA.

The host non-human vertebrate constant region herein is optionally the endogenous host wild-type constant region located at the wild type locus, as appropriate for the heavy or light chain. For example, the human heavy chain DNA is suitably inserted on mouse chromosome 12, suitably adjacent the mouse heavy chain constant region, where the vertebrate is a mouse.

In one optional aspect where the Vertebrate is a mouse, the insertion of the human antibody gene DNA, such as the human VDJ region is targeted to the region between the J4 exon and the Cμ locus in the mouse genome IgH locus, and in one aspect is inserted between coordinates 114,667,090 and 114,665,190, suitably at coordinate 114,667,091. In one aspect the insertion of the human antibody DNA, such as the human light chain kappa VJ is targeted into mouse chromosome 6 between coordinates 70,673,899 and 70,675,515, suitably at position 70,674,734, or an equivalent position in the lambda mouse locus on chromosome 16.

In one aspect the host non-human vertebrate constant region for forming the chimaeric antibody may be at a different (non endogenous) chromosomal locus. In this case the inserted human antibody DNA, such as the human variable VDJ or VJ region(s) may then be inserted into the non-human genome at a site which is distinct from that of the naturally occurring heavy or light constant region. The native constant region may be inserted into the genome, or duplicated within the genome, at a different chromosomal locus to the native position, such that it is in a functional arrangement with the human variable region such that chimaeric antibodies of the invention can still be produced.

In one aspect the human antibody DNA is inserted at the endogenous host wild-type constant region located at the wild type locus between the host constant region and the host VDJ region.

Reference to location of the variable region upstream of the non-human vertebrate constant region means that there is a suitable relative location of the two antibody portions, variable and constant, to allow the variable and constant regions to form a chimaeric antibody or antibody chain in vivo in the vertebrate. Thus, the inserted human antibody DNA and host constant region are in operable connection with one another for antibody or antibody chain production.

In one aspect the inserted human antibody DNA is capable of being expressed with different host constant regions through isotype switching. In one aspect isotype switching does not require or involve trans switching. Insertion of the human variable region DNA on the same chromosome as the relevant host constant region means that there is no need for trans-switching to produce isotype switching.

In the present invention, optionally host non-human vertebrate constant regions are maintained and it is preferred that at least one non-human vertebrate enhancer or other control sequence, such as a switch region, is maintained in functional arrangement with the non-human vertebrate constant region, such that the effect of the enhancer or other control sequence, as seen in the host vertebrate, is exerted in whole or in part in the transgenic animal. This approach is designed to allow the full diversity of the human locus to be sampled, to allow the same high expression levels that would be achieved by non-human vertebrate control sequences such as enhancers, and is such that signalling in the B-cell, for example isotype switching using switch recombination sites, would still use non-human vertebrate sequences.

A non-human vertebrate having such a genome would produce chimaeric antibodies with human variable and non-human vertebrate constant regions, but these are readily humanized, for example in a cloning step that replaces the mouse constant regions for corresponding human constant regions (eg, after the chimaeric antibody has been tested in the Assay Vertebrate).

In one aspect the inserted human IgH VDJ region comprises, in germline configuration, all of the V, D and J regions and intervening sequences from a human. Optionally, non-functional V and/or D and/or J gene segments are omitted. For example, VH which are inverted or are pseudogenes may be omitted.

In one aspect 800-1000 kb of the human IgH VDJ region is inserted into the non-human vertebrate IgH locus, and in one aspect a 940, 950 or 960 kb fragment is inserted. Suitably this includes bases 105,400,051 to 106,368,585 from human chromosome 14 (all coordinates refer to NCBI36 for the human genome, ENSEMBL Release 54 and NCBIM37 for the mouse genome, relating to mouse strain C57BL/6J).

In one aspect the inserted IgH human fragment consists of bases 105,400,051 to 106,368,585 from chromosome 14. In one aspect the inserted human heavy chain DNA, such as DNA consisting of bases 105,400,051 to 106,368,585 from chromosome 14, is inserted into mouse chromosome 12 between the end of the mouse J4 region and the Eμ region, suitably between coordinates 114,667,091 and 114,665,190, suitably at coordinate 114,667,091.

In one aspect the inserted human kappa VJ region comprises, in germline configuration, all of the V and J regions and intervening sequences from a human. Optionally, non-functional V and/or J gene segments are omitted.

Suitably this includes bases 88,940,356 to 89,857,000 from human chromosome 2, suitably approximately 917 kb. In a further aspect the light chain VJ insert may comprise only the proximal clusters of V segments and J segments. Such an insert would be of approximately 473 kb.

In one aspect the human light chain kappa DNA, such as the human IgK fragment of bases 88,940,356 to 89,857,000 from human chromosome 2, is suitably inserted into mouse chromosome 6 between coordinates 70,673,899 and 70,675,515, suitably at position 70,674,734.

In one aspect the human lambda VJ region comprises, in germline configuration, all of the V and J regions and intervening sequences from a human. Suitably this includes analogous bases to those selected for the kappa fragment, from human chromosome 2. Optionally, non-functional V and/or J gene segments are omitted.

All specific human antibody fragments described herein may vary in length, and may for example be longer or shorter than defined as above, such as 500 bases, 1 KB, 2K, 3K, 4K, 5 KB, 10 KB, 20 KB, 30 KB, 40 KB or 50 KB or more, which suitably comprise all or part of the human V(D)J region, whilst preferably retaining the requirement for the final insert to comprise human genetic material encoding the complete heavy chain region and light chain region, as appropriate, as described herein.

In one aspect the 3′ end of the last inserted human antibody sequence, generally the last human J sequence, is inserted less than 2 kb, preferably less than 1 KB from the human/non-human vertebrate (eg, human/mouse or human/rat) join region.

Optionally, the genome is homozygous at one, or both, or all three antibody loci (IgH, IgX and IgK).

In another aspect the genome may be heterozygous at one or more of the antibody loci, such as heterozygous for DNA encoding a chimaeric antibody chain and native (host cell) antibody chain. In one aspect the genome may be heterozygous for DNA capable of encoding 2 different antibody chains encoded by immunoglobulin transgenes of the invention, for example, comprising 2 different chimaeric heavy chains or 2 different chimaeric light chains.

In one embodiment in any configuration of the invention, the genome of the Vertebrate has been modified to prevent or reduce the expression of fully-endogenous antibody. Examples of suitable techniques for doing this can be found in WO2011004192, U.S. Pat. No. 7,501,552, U.S. Pat. No. 6,673,986, U.S. Pat. No. 6,130,364, WO2009/076464, EP1399559 and U.S. Pat. No. 6,586,251, the disclosures of which are incorporated herein by reference. In one embodiment, the non-human vertebrate VDJ region of the endogenous heavy chain immunoglobulin locus, and optionally VJ region of the endogenous light chain immunoglobulin loci (lambda and/or kappa loci), have been inactivated. For example, all or part of the non-human vertebrate VDJ region is inactivated by inversion in the endogenous heavy chain immunoglobulin locus of the mammal, optionally with the inverted region being moved upstream or downstream of the endogenous Ig locus. For example, all or part of the non-human vertebrate VJ region is inactivated by inversion in the endogenous kappa chain immunoglobulin locus of the mammal, optionally with the inverted region being moved upstream or downstream of the endogenous Ig locus. For example, all or part of the non-human vertebrate VJ region is inactivated by inversion in the endogenous lambda chain immunoglobulin locus of the mammal, optionally with the inverted region being moved upstream or downstream of the endogenous Ig locus. In one embodiment the endogenous heavy chain locus is inactivated in this way as is one or both of the endogenous kappa and lambda loci.

Additionally or alternatively, the Vertebrate has been generated in a genetic background which prevents the production of mature host B and T lymphocytes, optionally a RAG-1-deficient and/or RAG-2 deficient background. See U.S. Pat. No. 5,859,301 for techniques of generating RAG-1 deficient animals.

In one embodiment in any configuration of the invention, the human V, J and optional D regions are provided by all or part of the human IgH locus; optionally wherein said all or part of the IgH locus includes substantially the full human repertoire of IgH V, D and J regions and intervening sequences. A suitable part of the human IgH locus is disclosed in WO2011004192. In one embodiment, the human IgH part includes (or optionally consists of) bases 105,400,051 to 106,368,585 from human chromosome 14 (coordinates from NCBI36). Additionally or alternatively, optionally wherein the vertebrate is a mouse or the cell is a mouse cell, the human V, J and optional D regions are inserted into mouse chromosome 12 at a position corresponding to a position between coordinates 114,667,091 and 114,665,190, optionally at coordinate 114,667,091 (coordinates from NCBIM37, relating to mouse strain C57BL/6J).

In one embodiment of any configuration of a Vertebrate or cell (line) of the invention when the Vertebrate is a mouse, (i) the mouse comprises a transgenic heavy chain locus whose constant region comprises a mouse or rat Sμ switch and optionally a mouse Cμ region. For example the constant region is provided by the constant region endogenous to the mouse, eg, by inserting human V(D)J region sequences into operable linkage with the endogenous constant region of a mouse genome or mouse cell genome.

In one embodiment of any configuration of a Vertebrate or cell (line) of the invention when the Vertebrate is a rat, (i) the rat comprises a transgenic heavy chain locus whose constant region comprises a mouse or rat Sμ switch and optionally a rat Cμ region. For example the constant region is provided by the constant region endogenous to the rat, eg, by inserting human V(D)J region sequences into operable linkage with the endogenous constant region of a rat genome or rat cell genome.

In one embodiment of any configuration of a Vertebrate or cell (line) of the invention the lambda antibody transgene comprises all or part of the human Igλ locus including at least one human Jλ region and at least one human Cλ region, optionally C_{λ}6 and/or C_{λ}7. Optionally, the transgene comprises a plurality of human Jλ regions, optionally two or more of J_{λ}1, J_{λ}2, J_{λ}6 and J_{λ}7, optionally all of J_{λ}1, J_{λ}2, J_{λ}6 and J_{λ}7. The human lambda immunoglobulin locus comprises a unique gene architecture composed of serial J-C clusters. In order to take advantage of this feature, the invention in optional aspects employs one or more such human J-C clusters inoperable linkage with the constant region in the transgene, eg, where the constant region is endogenous to the non-human vertebrate or non-human vertebrate cell (line). Thus, optionally the transgene comprises at least one human J_{λ}-C_{λ} cluster, optionally at least J_{λ}7-C_{λ}7. The construction of such transgenes is facilitated by being able to use all or part of the human lambda locus such that the transgene comprises one or more J-C clusters in germline configuration, advantageously also including intervening sequences between clusters and/or between adjacent J and C regions in the human locus. This preserves any regulatory elements within the intervening sequences which may be involved in VJ and/or JC recombination and which may be recognised by AID (activation-induced deaminase) or AID homologues.

Where endogenous regulatory elements are involved in CSR (class-switch recombination) in the non-human vertebrate, these can be preserved by including in the transgene a constant region that is endogenous to the non-human vertebrate. In the first configuration of the invention, one can match this by using an AID or AID homologue that is endogenous to the vertebrate or a functional mutant thereof. Such design elements are advantageous for maximising the enzymatic spectrum for SHM (somatic hypermutation) and/or CSR and thus for maximising the potential for antibody diversity.

Optionally, the lambda transgene comprises a human Eλ enhancer. Optionally, the kappa transgene comprises a human Eκ enhancer. Optionally, the heavy chain transgene comprises a heavy chain human enhancer.

In one embodiment of any configuration of the invention the constant region of the or each antibody transgene is endogenous to the non-human vertebrate or derived from such a constant region. For example, the vertebrate is a mouse or the cell is a mouse cell and the constant region is endogenous to the mouse. For example, the vertebrate is a rat or the cell is a rat cell and the constant region is endogenous to the rat.

In one embodiment of any configuration of the invention the heavy chain transgene comprises a plurality human IgH V regions, a plurality of human D regions and a plurality of human J regions, optionally substantially the full human repertoire of IgH V, D and J regions.

In one embodiment of any configuration of the invention, the vertebrate or cell comprises a heavy chain further transgene, the further transgene comprising at least one human IgH V region, at least one human D region and at least one human J region, optionally substantially the full human repertoire of IgH V, D and J regions.

In one embodiment of any configuration of the invention, for the Antibody-Generating Vertebrate and/or Assay Vertebrate: —

(i) the heavy chain transgene comprises substantially the full human repertoire of IgH V, D and J regions; and

(ii) the vertebrate comprises substantially the full human repertoire of Igκ V and J regions and/or substantially the full human repertoire of IgX V and J regions.

An aspect provides a B-cell, hybridoma or a stem cell, optionally an embryonic stem cell or haematopoietic stem cell, derived from an Assay Vertebrate according to any configuration of the invention. In one embodiment, the cell is a B6, BALB/c, JM8 or AB2.1 or AB2.2 embryonic stem cell (see discussion of suitable cells, and in particular JM8 and AB2.1 cells, in WO2011004192, which disclosure is incorporated herein by reference).

In one aspect the ES cell is derived from the mouse BALB/c, C57BL/6N, C57BL/6J, 129S5, 129S7 or 129Sv strain.

In one aspect the non-human vertebrate is a rodent, suitably a mouse, and cells (cell lines) of the invention, are rodent cells or ES cells, suitably mouse ES cells.

The ES cells of the present invention can be used to generate animals using techniques well known in the art, which comprise injection of the ES cell into a blastocyst followed by implantation of chimaeric blastocysts into females to produce offspring which can be bred and selected for homozygous recombinants having the required insertion. In one aspect the invention relates to a transgenic animal comprised of ES cell-derived tissue and host embryo derived tissue. In one aspect the invention relates to genetically-altered subsequent generation animals, which include animals having a homozygous recombinants for the VDJ and/or VJ regions.

An aspect provides a method of isolating an antibody or nucleotide sequence encoding said antibody, the method comprising

(a) immunising (see e.g. Harlow, E. & Lane, D. 1998, 5^{th} edition, Antibodies: A Laboratory Manual, Cold Spring Harbor Lab. Press, Plainview, N.Y.; and Pasqualini and Arap, Proceedings of the National Academy of Sciences (2004) 101:257-259) an Antibody-Generating Vertebrate according to any configuration or aspect of the invention with a human target antigen such that the vertebrate produces test antibodies; and

(b) isolating from the vertebrate a test antibody that specifically binds to said antigen and/or a nucleotide sequence encoding at least the heavy and/or the light chain variable regions of said antibody;

optionally wherein the variable regions of said antibody are subsequently joined to a human constant region (eg, after testing of the antibody in an Assay Vertebrate of the invention). Such joining can be effected by techniques readily available in the art, such as using conventional recombinant DNA and RNA technology as will be apparent to the skilled person. See e.g. Sambrook, J and Russell, D. (2001, 3′d edition) Molecular Cloning: A Laboratory Manual (Cold Spring Harbor Lab. Press, Plainview, N.Y.).

Suitably an immunogenic amount of the human epitope or target antigen is delivered. The invention also relates to a method for detecting a human epitope or target antigen comprising detecting a test antibody produced as above with a secondary detection agent which recognises a portion of that antibody.

Isolation of the antibody in step (b) can be carried out using conventional antibody selection techniques, eg, panning for antibodies against antigen that has been immobilised on a solid support, optionally with iterative rounds at increasing stringency, as will be readily apparent to the skilled person.

As a further optional step, after step (b) the amino acid sequence of the heavy and/or the light chain variable regions of the antibody are mutated to improve affinity for binding to said antigen. Mutation can be generated by conventional techniques as will be readily apparent to the skilled person, eg, by error-prone PCR. Affinity can be determined by conventional techniques as will be readily apparent to the skilled person, eg, by surface plasmon resonance, eg, using Biacore™.

Additionally or alternatively, as a further optional step, after step (b) the amino acid sequence of the heavy and/or the light chain variable regions of a test antibody are mutated to improve one or more biophysical characteristics of the antibody, eg, one or more of melting temperature, solution state (monomer or dimer), stability and expression (eg, in CHO or E coli).

An aspect provides a test antibody of the invention, optionally for use in medicine, eg, for treating and/or preventing a medical condition or disease in a patient, eg, a human.

An aspect provides a nucleotide sequence encoding a test antibody of the invention, optionally wherein the nucleotide sequence is part of a vector. Suitable vectors will be readily apparent to the skilled person, eg, a conventional antibody expression vector comprising the nucleotide sequence together in operable linkage with one or more expression control elements.

An aspect provides a pharmaceutical composition comprising a test antibody of the invention and a diluent, excipient or carrier, optionally wherein the composition is contained in an IV container (eg, and IV bag) or a container connected to an IV syringe.

An aspect provides the use of a test antibody of the invention in the manufacture of a medicament for the treatment and/or prophylaxis of a disease or condition in a patient, eg a human.

In a further aspect the invention relates to humanised antibodies and antibody chains produced or assayed according to the present invention, both in chimaeric and fully humanised form, and use of said antibodies in medicine. The invention also relates to a pharmaceutical composition comprising such an antibody and a pharmaceutically acceptable carrier or other excipient.

Antibody chains containing human sequences, such as chimaeric human-non human antibody chains, are considered humanised herein by virtue of the presence of the human protein coding regions region. Fully human antibodies may be produced starting from DNA encoding a chimaeric antibody chain of the invention using standard techniques.

Methods for the generation of both monoclonal and polyclonal antibodies are well known in the art, and the present invention relates to both polyclonal and monoclonal antibodies of chimaeric or fully humanised antibodies produced in response to antigen challenge in non human-vertebrates of the present invention.

In a yet further aspect, chimaeric antibodies or antibody chains generated in the present invention may be manipulated, suitably at the DNA level, to generate molecules with antibody-like properties or structure, such as a human variable region from a heavy or light chain absent a constant region, for example a domain antibody; or a human variable region with any constant region from either heavy or light chain from the same or different species; or a human variable region with a non-naturally occurring constant region; or human variable region together with any other fusion partner. The invention relates to all such chimaeric antibody derivatives derived from chimaeric antibodies identified, isolated or assayed according to the present invention.

In a further aspect, the invention relates to use of an Assay Vertebrate of the present invention in the analysis of the likely effects of a drug or vaccine in the context of a human antibody variable region repertoire, the human epitope/target and the test antibody. This is useful for simulating the environment in vivo in human patients likely to receive the drug.

The invention also relates to a method for identification or validation of a drug or vaccine, the method comprising delivering the vaccine or drug to an Assay Vertebrate of the invention and monitoring one or more of: the immune response, the safety profile; the effect on disease. In one embodiment, the drug is a test antibody as herein defined; in another embodiment it is not, but the Assay Vertebrate contains both the drug and a test antibody. This is useful for assessing interactions, effect, performance, toxicity or PK (or any of the assay parameters mentioned above) of useful drugs (and drug candidates) in the presence of a test antibody; or conversely assessing this for a test antibody in the context of a known drug. In the latter, the drug may be a drug commonly found in patients of the type expected to receive the antibody as a therapeutic and/or prophylactic—which is useful when the antibody is intended to be a second-line (or subsequent) treatment in patients receiving the drug as a first-line (or earlier) treatment.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or an when used in conjunction with the term “comprising” in the claims and/or the specification may mean “one,” but it is also consistent with the meaning of “one or more,” “at least one,” and “one or more than one.” The use of the term or in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the feature in the context with which it is referred. The term “substantially” when referring to an amount, extent or feature (eg, “substantially identical” or “substantially the same”) includes a disclosure of “identical” or “the same” respectively, and this provides basis for insertion of these precise terms into claims below.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term or combinations thereof′ as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non limiting exemplification.

### EXAMPLES

The following examples will be useful for demonstrating the present invention. Example 3 is a fully-worked example and data from this is provided below as a non-limiting illustration of how to derive ES cells for use in the present invention.

### Example 1

### Generation of Transgenic Antibody-Generating Mouse

A transgenic mouse is generated using ES cell technology and genetic manipulation to introduce human antibody heavy chain and kappa chain V, D and J segments operatively connected directly 5′ of endogenous mouse heavy and kappa constant regions respectively. Mouse mu switch and mu constant and gamma regions are provided in the heavy chain transgenic locus thus produced. Endogenous, mouse heavy chain and kappa chain expression are inactivated; mouse lambda chain expression is typically 5% or less so inactivation is optional. The human antibody gene segments are introduced into a mouse ES cell using homologous recombination and/or recombinase mediated cassette exchange (RMCE) as is known in the art. Human DNA can be manipulated using BAC and recombineering technology as known in the art. BACs containing human antibody gene DNA is obtainable from Invitrogen. A suitable ES cell is a 129, AB2.1 or AB2.2 cell (obtainable from Baylor College of Medicine).

The transgenic ES cells are then implanted into a blastocyst from a foster mouse mother (eg, a 129 or C57BL/6N mouse strain). Heavy chain and kappa chain lines can be produced and crossed to provide an Antibody-Generating mouse bearing homozygous transgenic heavy and kappa chains with human variable regions (HK mouse).

Using a similar protocol, a lambda chain line is produced and by crossing a HKL mouse is generated bearing homozygous transgenic heavy, lambda and kappa chains with human variable regions

Further guidance is disclosed in WO2011004192, U.S. Pat. No. 7,501,552, U.S. Pat. No. 6,673,986, U.S. Pat. No. 6,130,364, WO2009/076464 and U.S. Pat. No. 6,586,251, the disclosures of which are incorporated herein by reference in their entirety.

### Isolation of Test Antibody

Using a human target antigen in a suitable injection medium (eg, including an adjuvant such as Freunds or Titermax™), the HK Antibody-Generating mouse is immunised. RIMMS is a suitable immunisation protocol, or any other standard immunisation protocol.

A test antibody is isolated that comprises human variable regions and binds the human antigen with desired affinity. Affinity is tested using surface plasmon resonance (eg, using Biacore™)

### ES Cell Recovery & Production of Assay Mice:

The HK Antibody-Generating mouse is crossed with a mouse of the same genetic background; in this example, the mice are of a 129 background and have essentially the same immune gene repertoire. ES cells are isolated from an embryo resulting from the cross. Such ES cells (H/H; K/K—ie, homozygous for the antibody transgenes) are further used to knock-out an endogenous target gene that is orthologous or homologous to the human target used previously for immunisation; while the gene for the human target is knocked into the ES cell genome. In one embodiment, using homologous recombination and a vector harbouring the DNA for the human target, the endogenous gene is replaced by the human gene so that the genome comprises a knock-out for the endogenous gene and a knock-in for the human gene. A suitable vector has the human DNA flanked by homology arms which are mouse sequences immediately 5′ and 3′ of the endogenous target DNA in the ES cell genome.

ES cell chimera are generated and used for production of mice demonstrating germline transmission (F1 mice: H/HA; K/KA, where H/HA=heterozygous−transgenic heavy chain locus+inactivated endogenous heavy chain locus and K/KA=heterozygous−transgenic kappa chain locus+inactivated endogenous kappa locus; KI or KO/+). F2 mice (H/HA or H; K/KA or K; KO/KO or KI/KI) are then generated by crossing the F1 mice to produce [H/H+K/K+KI/KI] or [H/H+K/K+KI/KI+KO/KO] Assay Mice.

### Antibody Testing

The Test Antibody is injected into an Assay Mouse and one or more of the following is determined: —

pharmacodynamics of said antibody (or a metabolite or derivative thereof produced by the Assay Mouse), pharmacokinetics of said antibody, activity of said antibody, clearance of said antibody, distribution of said antibody, toxicology of said antibody, a physico-chemical characteristic or effect of said antibody, a binding characteristic of said antibody, a biological characteristic or effect of said antibody, a physiological characteristic or effect of said antibody, a pharmaceutical characteristic or effect of said antibody, and interaction of said antibody with another protein or substance inside the Assay Mouse. The skilled person will be fully conversant with standard techniques for carrying out such assays.

The test antibody bears constant regions that are species-matched for the Assay Mouse and the antibody is seen as “self” by the mouse and thus tolerated. Thus, issues of anti-test antibody reaction are not encountered which would otherwise hamper the assay. By matching the Antigen-Generating Vertebrate and Assay Vertebrate (and thus matching the constant region of the test antibody), the present invention provides for pre-clinical and clinical assay testing with more accuracy and less risk of anti-antibody interference of data sets. This allows for better selection of lead candidates for progression into clinical development and drug production.

### Humanisation of Test Antibody

The method is conducted for a panel of test antibodies. A lead candidate is chosen according to affinity for binding the human target and one or more of the assay parameters discussed above.

Using recombinant DNA technology, as is standard, the mouse constant regions of the lead candidate are replaced with corresponding human constant regions to produce a fully-human antibody that binds the human target.

### Example 2

Example 1 is carried out with the exception that IPS cell (induced pluripotent cell) generation is carried out instead of ES cell recovery.

### IPS Cell Recovery:

Mouse embryonic fibroblasts (MEF) which are isolated from embryos derived from crossing of parents carrying the antibody transgenes (HK or HKL mice) are induced to IPS cells. The IPS cells can also be directly generated from other somatic cells from mice carrying the antibody transgenes.

Such iPS cells (H/H; K/K) are further used to knock-in the human target gene (and optionally knock out the endogenous mouse orthologue or homologue). IPS chimeras are generated and used for production of germline transmission mice (F1 mice: H/HA; K/KA; KI or KO/+). F2 mice (H/HA or H; K/KA or K; KO/KO or KI/KI) are then generated by crossing the F1 mice.

### Example 3

### Derivation of ES Cells from Antibody-Generating Non-Human Vertebrates

The aim of this experiment was to test our protocol for ES Cell derivation, to see if we could produce ES cells from mice we have created.

### Parental Mice Cross

Mice were heterozygous for S3F (ie, 53F/+). S3F denotes a transgenic IgH locus comprising a human gene segment repertoire V_{H}2-5, V_{H}7-4-1, V_{H}4-4, V_{H}1-3, V_{H}1-2, V_{H}6-1, D1-1, D2-2, D3-9, D3-10, D4-11, D5-12, D6-13, D1-14, D2-15, D3-16, D4-17, D5-18, D6-19, D1-20, D2-21, D3-22, D4-23, D5-24, D6-25, D1-26, D7-27, J_{H}1, J_{H}2, J_{H}3, J_{H}4, J_{H}5 and J_{H}6 (in 5′ to 3′ order) upstream of a mouse heavy chain constant region. The “+” indicates wild type mouse IgH allele.

(Genetic back ground is: 12957/SvEvBrd, C57BL/6Brd-Tyr^{c-Brd})

The parental cross was:

“KMSP95.1b” Male (53F/+)×“KMSP95.2g” Female (53F/+)

We used the protocol detailed below and obtained 8 Blastocysts.

### ESC Derivation Media:

Knockout-DMEM (Gibco)

15% Knockout serum replacement (Gibco)

5% ESC-grade FCS (Gibco)

1×NEAA (Gibco)

2 mM Glutamine (Gibco)

0.1 mM 2-Mercaptoethanol

3000 U/MI LIF (ESGRO)
1. Flush e3.5 embryos from uterus and plate onto a feeder plate (6 Well plates can be convenient for picking colonies later) with above mentioned medium.
2. Don't disturb for next 48 hours
3. Medium change every other day. Around day 5, look for the Inner Cell Mass outgrowth, or ICM.
4. Pick colonies around day 7-10, trypsinise into a single cell suspension in a round bottom 96 well plate, and plate onto 6 well plates. Briefly pick the outgrowths into a 96 well containing 25-30 □l 0.25% trypsin (Sigma) solution, incubate for 3-4 mins. Using a 10 □l pipette desegregate the ICM gently into smaller cellular aggregates of three or four cells. Transfer the contents form the 96 well onto freshly prepared 6 well plates, each well going to one 6 well.
5. Inspect the plates daily. After about 2 days primary colonies of cells will become visible and may have one of several morphologies: trophoblast-like cells, epithelium-like cells, endoderm-like cells, ES cell-like cells which are what we are looking for. If the plate contains clumps with ES cell morphology which are the majority of cells, passage the cells following normal protocol for maintenance, trypsinise every other day. If there are very few cells with ES cell morphology, these can be picked and trypsinised as described in step 4. Once cells have been expanded to 2×10 cm dishes they can be frozen for storage.
6. Freeze cells following a standard freezing protocol.
7. When frozen cells are thawed, the can be placed into the usual ES cell culture media KO-DMEM+15% Serum+1000 U/MI LIF

### Identification of ES Cells

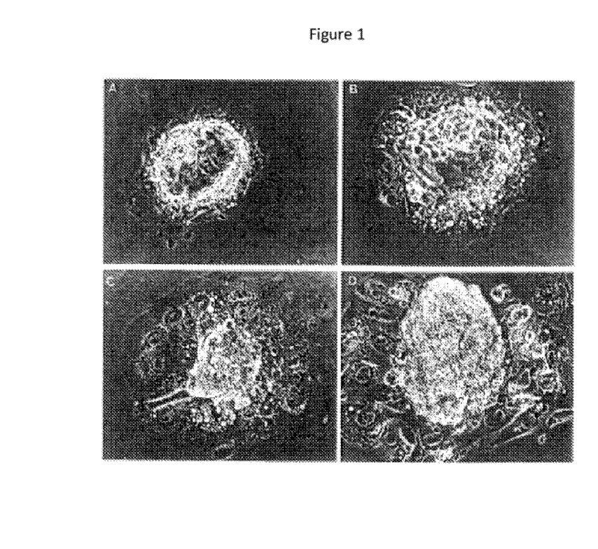

In step 3, ICM of the desired morphology can be seen by the illustrative example in box D of FIG. 1. FIG. 1 shows the progressive changes in morphology of cultured blastocysts (taken from “Manipulating the Mouse Embryo”, 3^{rd} Edition, A Nagy et al, Cold Spring Harbor Laboratory Press, 2003; FIG. 8.2 of that text).

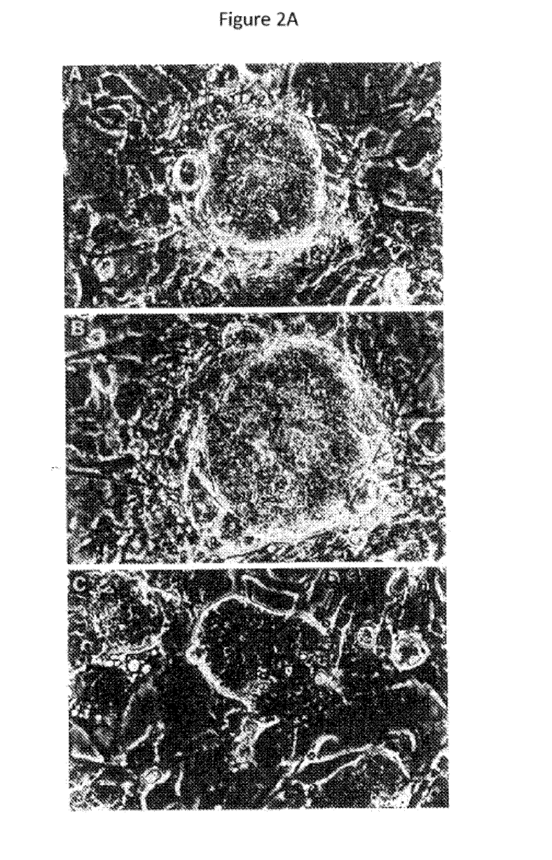

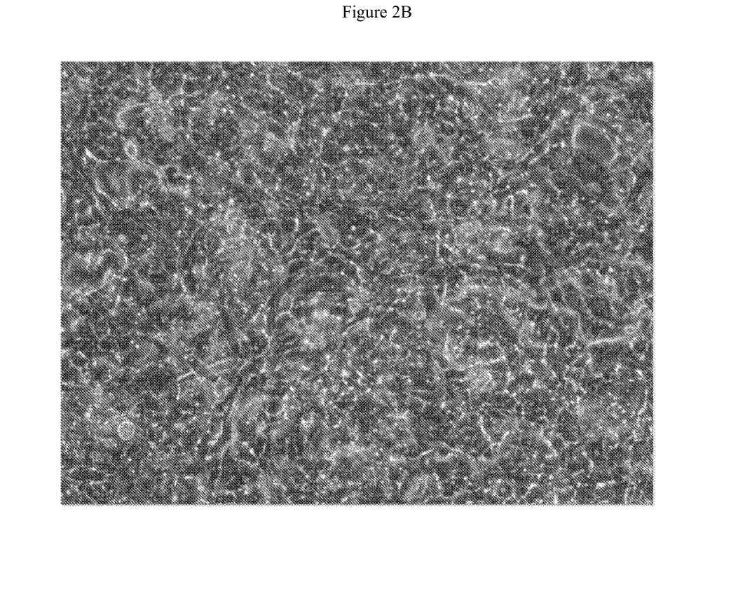

In step 5, ES cells have characteristic morphology, as will be known by the skilled person. An illustration is shown in FIG. 2A (taken from “Manipulating the Mouse Embryo”, 3^{rd} Edition, A Nagy et al, Cold Spring Harbor Laboratory Press, 2003; FIG. 8.4 of that text). Nagy et al provides a description as follows: box A shows a colony of stem cells 2 days after disaggregation on the ICM; box B shows the same colony 2 days later. The colony remains composed of a homogeneous population of stem cells and no overt cellular differentiation has occurred. Stem cells are comparatively small, typically have a large clear nucleus containing one or more prominent nucleoli and are tightly packed within the multilayered primary colony. In box C, the colony was subcultured into fresh a feeder well. Within 2 days numerous small nests of stem cells appeared in culture.

Additionally or alternatively to the use of morphology to look for ES cells, the skilled person will be aware of the use of ES cell markers (eg, Nanog and Oct4) for this purpose. Oct4 and Nanog are transcription factors required to maintain the pluripotency and self-renewal of embryonic stem (ES) cells.) as described in the following paper: Nature Genetics 38, 431-440 (2006); Published online: 5 Mar. 2006; | doi:10.1038/ng1760; “The Oct4 and Nanog transcription network regulates pluripotency in mouse embryonic stem cells”.

In the present example we did not use these markers for our work; we only look at the morphology. Following this method we obtained 3 separate clones which when assessed under the microscope showed the morphological characteristics of ES cells (see FIG. 2B). Stem cells are comparatively small, typically have a large clear nucleus containing one or more prominent nuclei and are tightly packed within the multi-layered primary colony.

Once the clones were expanded onto 2×10 cm plates they were frozen following a normal freezing protocol. Briefly the cells are trypsinised as described above this time incubating for 20 mins, the trypsin is inactivated using an equal volume of ES cell media, the cells are pipetted to separate colonies. The cell suspension is collected and centrifuged, any supernatant is removed and the cells re-suspended in ES cell media. An equal volume of freeze media is added (60% DMEM, 20% FBS, 20% DMSO (Sigma), freshly prepared), 1 ml is aliquoted into pre-labelled sterile freezing vials, 6 vials per clone.

The clones were named KX01.1, KX01.2 and KX01.3.

### Genotyping Analysis

A small volume of each clone was kept aside to be used for genotyping analysis to ensure the ES cells obtained carried the same gene as the parent animals used to produce the blastocysts. Alongside genotyping we tested for the Y chromosome as our ES cells should ideally be male. Male ES cells are preferred over female ES cells because they offer much better rates of germ line transmission:

(i) They are much more stable in culture. Female [XX] mouse ES cells have two active X-chromosomes, meaning that the dose of X-to-autosomal gene products is 1:1, while in all other cell types it is 1:2 because of X-inactivation. This unusual 1:1 gene dosage situation is not well tolerated and often one of X-chromosomes is lost or deleted.

(ii) Most reported germ line transmission events come from male chimaeras because the male XY ES cells will often convert female embryos into phenotypic males. As a result the majority of chimaeras born [75-80%] following the injection of male ES cells into blastocysts are phenotypic males which transmit their male ES cell-derived genomes. Even if the injected embryo is female, male ES cells can convert the chimera into a fully functional fertile male.

(iii) Male chimaeras can be extensively and quickly bred, producing 100s of progeny in a matter of months if required. So even low levels of contribution to sperm can be rapidly detected.

(iv) Although the injection of female ES cells can result in germ line transmission, this is only possible through female chimaeras. It is not possible to breed females extensively, because litters are limited in size and frequency, thus low level germ-line chimaerism will not be reliably detected.

### Genotyping Protocols

ES cell genotyping was conducted using the following protocol, this was to ensure that the ES cells obtained carried the same gene as the parent animals used to obtain the blastocysts.

### ES Cell Digestion

Remove media
Wash with 500 μl PBS
Use the ES cell lysis buffer (50 mM Tris, 50 mM EDTA, 1% SDS, 100 mM NaCl) Add PK powder from freezer (Sigma P8044-5g) to create a 1 mg/ml concentration
Add 500 μl of PK Lysis buffer to each well.
Seal plate with tape and put it in a plastic container that also contains paper towel soaked with water.
   This will keep the wells from drying out
Incubate at 55° C. overnight.
Move samples into eppendorf tubes and add equal volumes of isopropanol (in this case add 500 μl)
Centrifuge for 10 mins at 13000 rpm to form a DNA pellet.
Pour off supernatant and added equal volumes (in this case 1 ml of 70% ethanol) to wash.
Add 100 μl of Water to resuspend the DNA, ˜1 μl of DNA is used.

Appropriate primer sequences were used in the PCR reaction using the appropriate PCR cycle for this preparation.

The same digested DNA was used for the Y chromosome PCR which used the following primer sequences:

Sry3
(SEQ ID NO: 1)
ATGGAGGGCCATGTCAAGCGCCCCATGAA
Sry5
(SEQ ID NO: 2)
TTGCTGGTTTTTGGAGTACAGGTGTGCAGC

The protocol below was followed for the Y chromosome PCR:

2. After lysis, 1:10 dilution, take 1 ul for PCR reaction;

3. PCR system:

| | | |
|---|---|---|
| DNA | 1 | ul |
| Primer Forward concentration | 0.1 | uM |
| Primer Reverse concentration | 0.1 | uM |
| 2x Mongo Mix | 10 | ul |
| Water | 9 | ul |

4. PCR reaction

Close Lid 105.0° C. Auto Tube Pressure

Hot Start Automatic 94.0° C. 00:02.00

Start Cycle JUNCTION PCR 30 times

Denaturation 94.0° C. 00:00.30

Annealing 56.0° C. 00:00.30

Elongation 68.0° C. 00:01.00

End Cycle ‘JUNCTION PCR’

Elongation 68.0° C. 00:10.00

### Results Obtained:

The genotyping indicated the following results

KX01.1: S3F/S3F

KX01.2: S3F/+

KX01.3: S3F/+

Y Chromosome PCR result:

All clones resulted in female ES cells.

### Example 4

### General KO and KI Strategies

Generating monoclonal antibodies in animal systems is well documented and it has resulted in the successful development of numerous therapeutic antibodies. The process leading to the generation of a high-quality monoclonal antibody against a given target involves importantly the immunogenicity of the given antigen or target. For example, a target which is highly conserved between humans and the animal host, mouse for example, will result in a poor immune response due to self-tolerance and thus it will be difficult to generate good antibody leads against the human target using the conventional approach.

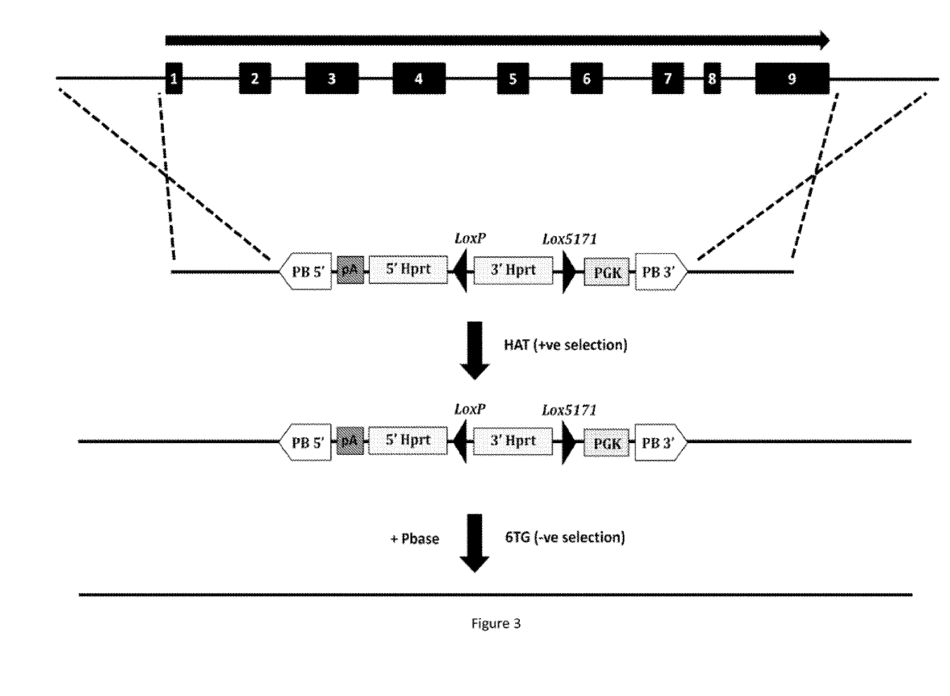

There are several ways to improve the immune response and breaking the immune tolerance using adjuvants, various toll-like receptor agonists, altering the immunisation regime and using target-specific gene knock-out (KO) mice lines for immunisation. The latter approach of generating specific knock-out mice is a convenient approach for overcoming the limitation of immune tolerance to human targets. Generating knock-out mice however could be both time consuming and costly. To this end, establishment of a streamlined methodology for generating specific KO mice is essential. The methodology described herein for generating specific KO mice allows exact deletion of the gene of interest and without leaving behind DNA scar or any exogenous DNA material normally left behind using traditional gene KO methodologies. The KO methodology is depicted schematically in FIG. 3.

Once a therapeutic antibody lead has been generated, it is important to test it on a relevant preclinical model and often such a model may not exist. Where therapeutic antibodies have been raised against human targets and which do not cross-react with the murine counterpart, incorporating the human target into the murine system using a knock-in (KI) approach can establish a murine preclinical model. To supplement the preclinical model further and depending on the therapeutic target, it could be beneficial to KI the human target and any additional human interacting partners to better reflect the natural cellular protein interactions in the animal model. In the case of a receptor, CD40 for example, its interacting ligand, CD40L, could also be knocked-in the murine host. There are several strategies one could take to KI human targets into a murine model. As described above for generating a KO mice, the murine target could be initially KO in its entirety depending on the size of the gene and the human target could be KI (eg, prior to the excision of the landing pad in the method described below). An alternative approach would be to humanise by knocking-in only the relevant human gene segment known to be involved in antibody binding or carry out an exon-specific knock-in. Such an approach will maintain the cis-regulatory elements, endogenous promoter and any signal peptides from the mouse or other model organism required for cell signalling thus maintaining the gene expression of the in-coming human gene segment under the same control as the endogeneous wild-type allele. This in turn will provide a platform for conducting preclinical studies. Also, this will be a useful alternative to KO/KI where the target gene is excessively large and thus where it may be difficult, time-consuming or costly to carry out a complete gene KO or KI using gene targeting in ES cells. Furthermore, knocking-in only part of human genes is less likely to alter gene regulation and in-turn the gene expression profile in the model organism.

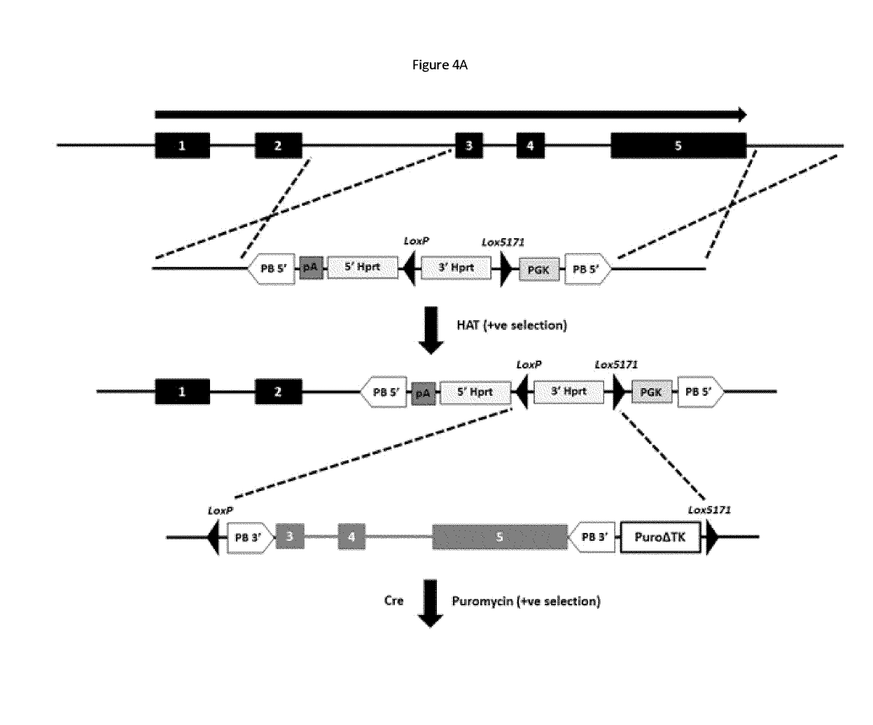

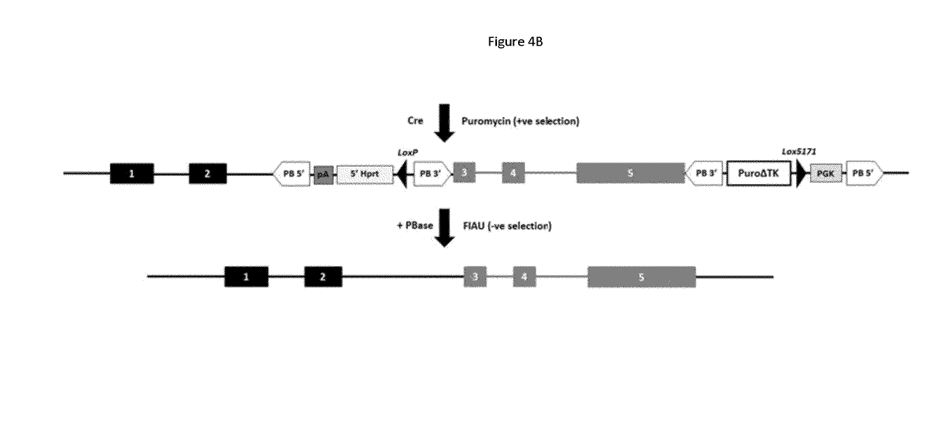

The methodology described herein is designed to expedite the process of creating precise gene KI, which is easily amenable to alteration to suit the skilled person's requirement for KI. For example the method could be used to KI a single human exon, several exons or the entire human gene. Exemplary KI methodology is depicted schematically in FIGS. 4A and 4B.

### Example 5

### Exemplary KO Method

FIG. 3 shows a schematic representation of a precise gene knock-out strategy for use in the present invention. A targeting vector (eg, a bacterial artificial chromosome) is designed against a target gene of interest using homology arms flanking the region destined for deletion. The features included in the targeting vector include HPRT gene split with loxP site and a mutant loxP site, lox5171, under the regulation of PGK promoter, which is flanked by PBase 5′ and 3′ LTR forming a PiggyBac transposon. Targeting is achieved by homologous recombination in ES cells whereby targeted clones are positively selected on hypoxanthine aminopterin thymidine (HAT) medium. The genomic region within the homology arm will be knocked-out and replaced by the transposon. The transposon could then be conveniently removed by transiently expressing the transposase and negatively selecting for the excision of the transposon using 6-thioguanine (6TG). This will leave behind a precise deletion unmarked with any exogenous DNA material. Note: The lox sites can instead be retained as part of a “landing pad” for subsequent targeting of a human gene or gene portion (eg, exon). Thus, the lox sites could be used as a base for knocking-in gene of interest using recombinase-mediated cassette exchange (RMCE) and acts as a landing pad for in-coming DNA (shown further in the following KI example).

### Example 6

### Exemplary KI Method

FIGS. 4A-4B show schematic representations of a precise gene knock-in strategy whereby exons 3-5 of a mouse gene (black boxes) is replaced with the human equivalent (grey boxes). A PiggyBac transposon is knocked-in a defined region within the gene of interest using homologous recombination. Targeted clones are positively selected on hypoxanthine aminopterin thymidine medium. Targeting of the transposon will KO the region of the gene that is required for knocking-in the human equivalent and it acts as a landing pad for knocking-in any DNA material of interest. The equivalent human exons 3-5 are knocked-in via the lox sites using RMCE. This creates two independent functional transposon elements, each flanked by 5′ and 3′ PB LTRs, and which are conveniently excised simultaneously by transiently expressing PBase transposase in ES cells correctly targeted with the initial landing pad. Removal of the transposons and thus the generation of a precise exon-specific gene KI in ES cell clones is negatively selected with 1-(2-deoxy-2-fluoro-D-arabinofuranosyl)-5 iodouracil (FIAU). The inserted human exon(s) precisely replace the mouse (non-human vertebrate) sequence and are conveniently placed under endogenous mouse regulatory control.

As is known in the art, several non-human vertebrate ES cells are available for use in these methods, wherein the engineered (KO and/or KI) ES cell can be implanted into a blastocyst and transferred to a donor mouse or other appropriate non-human vertebrate surrogate. Non-human vertebrates bearing the desired KO/KI are then developed from the implanted blastocyst and progeny thereof.

All publications cited herein are hereby incorporated by reference.

1. A method of assaying a test antibody comprising human variable regions that bind to a human epitope, wherein the antibody is isolated from a first transgenic non-human vertebrate wherein said vertebrate is designated an Antibody-Generating Vertebrate, optionally a mouse or a rat, following immunisation with an antigen bearing said human epitope, and optionally subsequent derivatisation or maturation of said antibody, the vertebrate comprising one or more transgenic antibody loci encoding said variable regions, and the transgenic vertebrate having an immune system comprising proteins encoded by an immune gene repertoire, said immune gene repertoire comprising said transgenic antibody loci, the method comprising
(a) providing a second transgenic non-human wherein said vertebrate is designated as an Assay Vertebrate, optionally a mouse or a rat, that is a modified version of said first transgenic non-human vertebrate, wherein the Assay Vertebrate comprises
(i) an immune system comprising proteins encoded by substantially the same immune gene repertoire as the Antibody-Generating Vertebrate;
(ii) a genome comprising a knock-in of said human epitope, so that the Assay Vertebrate is capable of expressing an antigen bearing said human epitope; and
(iii) optionally wherein said genome has a knock-out of an endogenous non-human vertebrate epitope that is an orthologue or homologue of said human epitope, wherein said Assay Vertebrate cannot express an antigen bearing said endogenous epitope;
(b) introducing said antibody into the Assay Vertebrate; and
(c) assaying the effect or behaviour of said antibody in said Assay Vertebrate.

2. The method of claim 1, wherein the Antibody-Generating Vertebrate and Assay Vertebrate have substantially identical genomes with the exception that the Assay Vertebrate genome comprises said knock-in.

3. The method of claim 1, wherein the Antibody-Generating Vertebrate and Assay Vertebrate genomes comprise said knock-out.

4. The method of claim 1, wherein in step (c) said assaying is assay of one or more selected from the group consisting of: pharmacodynamics of said antibody, pharmacokinetics of said antibody, activity of said antibody, clearance of said antibody, distribution of said antibody, toxicology of said antibody, a physico-chemical characteristic or effect of said antibody, a binding characteristic of said antibody, a biological characteristic or effect of said antibody, a physiological characteristic or effect of said antibody, a pharmaceutical characteristic or effect of said antibody, and interaction of said antibody with another protein or substance inside the Assay Vertebrate; and immunogenicity of the antibody.

5. The method of claim 1, wherein the Antibody-Generating Vertebrate is a genetic parent or grandparent of the Assay Vertebrate.

6. The method of claim 1, wherein the Assay Vertebrate is derived from a somatic cell of said Antibody-Generating Vertebrate; optionally wherein the Assay Vertebrate is derived from an IPS cell that is derived from said Antibody-Generating Vertebrate.

7. An assay kit comprising an Antibody-Generating Vertebrate and Assay Vertebrate as defined in claim 1, and optionally a test antibody.

8. A non-human, optionally a mouse or a rat, Assay Vertebrate comprising
(i) one or more transgenic antibody loci encoding human variable regions;
(ii) an immune system comprising proteins encoded by an immune gene repertoire, said immune gene repertoire comprising said transgenic antibody loci;
(iii) a genome comprising a knock-in of a human epitope, so that the Assay Vertebrate is capable of expressing an antigen bearing said human epitope; and
(iv) a genome knock-out of the endogenous non-human vertebrate epitope that is an orthologue or homologue of said human epitope, wherein said Assay Vertebrate cannot express an antigen bearing said endogenous epitope; and
(v) optionally a test antibody inside said Assay Vertebrate, wherein the antibody comprises human variable regions that can bind said human epitope, said antibody having been generated in an Antibody-Generating Vertebrate as defined in claim 1, and optionally having undergone subsequent derivatisation or maturation of said antibody,

9. A method of generating a non-human Assay Vertebrate, optionally a mouse or a rat, for assaying the effect or behaviour of a test antibody comprising human variable regions and which binds a human epitope, the method comprising
(a) obtaining a non-human vertebrate child ES cell whose genome is a genetic cross between:
(i) the genome of a first genetic parent that is a non-human Antibody-Generating Vertebrate whose genome encodes said test antibody, the Antibody-Generating Vertebrate comprising one or more transgenic antibody loci encoding antibodies comprising human variable regions, and the Antibody-Generating Vertebrate having an immune system comprising proteins encoded by an immune gene repertoire, said immune gene repertoire comprising said transgenic antibody loci; and
(ii) the genome of a second genetic parent that is a non-human vertebrate of the same species, optionally the same strain, as said Antibody-Generating Vertebrate, the second parent having an immune system encoded by substantially the same immune gene repertoire as the first parent;
(b) producing in vitro a modified child ES cell with a knock-in of the human epitope by introducing into the genome of the child ES cell a nucleotide sequence encoding said human epitope and optionally knocking-out of the genome an endogenous non-human vertebrate epitope that is an orthologue of said human epitope; and
(c) developing a non-human child vertebrate from said modified child ES cell, wherein an Assay Vertebrate is obtained that expresses said human epitope; and
(d) optionally producing a progeny of said Assay Vertebrate by genetic crossing, wherein said progeny comprises substantially the same immune gene repertoire as said Assay Vertebrate in addition to the human epitope knock-in, and optionally the knock-out.

10. The method of claim 9, wherein the first and second genetic parents (a) (i) and (ii) are of the same non-human vertebrate, optionally a mouse or a rat, strain.

11. The method of claim 10, wherein the first and second genetic parents are related as (a) siblings, (b) parent and child, (c) parent and grandchild, (d) cousins or (e) uncle/aunt and nephew/niece.

12. A method of generating a non-human Assay Vertebrate, optionally a mouse or a rat, for assaying the effect or behaviour of a test antibody comprising human variable regions and which binds a human epitope, the method comprising:
(a) obtaining a non-human vertebrate child ES cell from a somatic cell, wherein optionally said cell is an IPS cell, of a non-human Antibody-Generating Vertebrate whose genome encodes said test antibody, the Antibody-Generating Vertebrate comprising one or more transgenic antibody loci encoding antibodies comprising human variable regions, and the Antibody-Generating Vertebrate having an immune system comprising proteins encoded by an immune gene repertoire, said immune gene repertoire comprising said transgenic antibody loci;
(b) producing a modified child ES cell with a knock-in of the human epitope by introducing into the genome of the child ES cell a nucleotide sequence encoding said human epitope and optionally knocking-out of the genome an endogenous non-human vertebrate epitope that is an orthologue of said human epitope; and
(c) developing a non-human child vertebrate from said modified child ES cell, wherein an Assay Vertebrate is obtained that expresses said human epitope; and
(d) optionally producing a progeny of said Assay Vertebrate by genetic crossing, wherein said progeny comprises substantially the same immune gene repertoire as said Assay Vertebrate in addition to the human epitope knock-in, and optionally the knock-out.

13. The method of claim 12, wherein the IPS cell is a mouse embryonic fibroblast cell.

14. A method of generating a non-human Assay Vertebrate, optionally a mouse or rat, for assaying the effect or behaviour of a test antibody comprising human variable regions and which binds a human epitope, the method comprising
(a) providing an ES cell derived from an Antibody-Generating Vertebrate whose genome encodes said test antibody, the Antibody-Generating Vertebrate comprising one or more transgenic antibody loci encoding antibodies comprising human variable regions, and the Antibody-Generating Vertebrate having an immune system comprising proteins encoded by an immune gene repertoire, said immune gene repertoire comprising said transgenic antibody loci;
(b) introducing into the genome of the ES cell a nucleotide sequence encoding said human epitope and optionally knocking-out of the genome an endogenous non-human vertebrate epitope that is an orthologue of said human epitope; and
(c) developing a non-human child vertebrate from said modified ES cell, wherein an Assay Vertebrate is obtained that expresses said human epitope; and
(d) optionally producing a progeny of said Assay Vertebrate that is homozygous for said knock-in, wherein said progeny comprises substantially the same immune gene repertoire as said Assay Vertebrate in addition to the human epitope knock-in, and optionally the knock-out.

15. A method of assaying a test antibody comprising human variable regions that bind to a human epitope, wherein the antibody is isolated from a first transgenic non-human vertebrate, optionally a mouse or rat, designated as an Antibody-Generating Vertebrate following immunisation with an antigen bearing said human epitope, and optionally subsequent subsequent derivatisation or maturation of said antibody, the vertebrate comprising one or more transgenic antibody loci encoding said variable regions, the method comprising:
(a) providing a second transgenic non-human vertebrate, optionally a mouse or rat, designated as an Assay Vertebrate, that is a modified version of said first transgenic non-human vertebrate, wherein the Assay Vertebrate has substantially the same genome as the Antibody-Generating Vertebrate, with the exception that:
(i) the Assay Vertebrate genome comprises a knock-in of said human epitope, so that the Assay Vertebrate is capable of expressing an antigen bearing said human epitope; and
(ii) optionally, wherein said genome has a knock-out of an endogenous non-human vertebrate epitope that is an orthologue or homologue of said human epitope, wherein said Assay Vertebrate cannot express an antigen bearing said endogenous epitope;
(b) introducing said antibody into the Assay Vertebrate; and
(c) assaying the effect or behaviour of said antibody in said Assay Vertebrate.

16. The method of claim 15, wherein the Antibody-Generating Vertebrate and Assay Vertebrate genomes comprise said knock-out.

17. An assay kit comprising an Antibody-Generating Vertebrate and Assay Vertebrate as defined in claim 15, and optionally a test antibody.

18. A non-human, optionally a mouse or rat, Assay Vertebrate comprising:
(i) one or more transgenic antibody loci encoding human variable regions;
(ii) a genome comprising a knock-in of a human epitope, so that the Assay Vertebrate is capable of expressing an antigen bearing said human epitope; and
(iii) a genome knock-out of the endogenous non-human vertebrate epitope that is an orthologue or homologue of said human epitope, wherein said Assay Vertebrate cannot express an antigen bearing said endogenous epitope; and
(iv) optionally a test antibody inside said Assay Vertebrate, wherein the antibody comprises human variable regions that can bind said human epitope, said antibody having been generated in an Antibody-Generating Vertebrate as defined in claim 1, optionally with subsequent derivatisation or maturation to produce said antibody.

19. The method of claim 1, wherein the human epitope is a human CD40 ligand or human CD40 epitope; optionally wherein the knock-in is a knock-in of human CD40 ligand or human CD40.

20. The vertebrate of claim 18, wherein the human epitope is a human CD40 ligand or human CD40 epitope; optionally wherein the knock-in is a knock-in of human CD40 ligand or human CD40.

21. The kit of claim 17, wherein the human epitope is a human CD40 ligand or human CD40 epitope; optionally wherein the knock-in is a knock-in of human CD40 ligand or human CD40.
